SEQUENCE LISTING (42 sequences)

SEQ ID NO: 1 - DNA, 20 bp, Artificial Sequence
  PRIMER
cctcaaagctcagcgtgtcc

SEQ ID NO: 2 - DNA, 20 bp, Artificial Sequence
  PRIMER
gagctcacttttgcgccaag

SEQ ID NO: 3 - DNA, 19 bp, Artificial Sequence
  PRIMER
ggcccttctccaggacaga

SEQ ID NO: 4 - DNA, 20 bp, Artificial Sequence
  PRIMER
gctgatcatggctgggttgt

SEQ ID NO: 5 - DNA, 22 bp, Artificial Sequence
  PRIMER
gaaatcgtgcgtgacatcaaag

SEQ ID NO: 6 - DNA, 22 bp, Artificial Sequence
  PRIMER
tgtagtttcatggatgccacag

SEQ ID NO: 7 - DNA, 20 bp, Artificial Sequence
  PRIMER
tcctggctcaaaaagcagtt

SEQ ID NO: 8 - DNA, 20 bp, Artificial Sequence
  PRIMER
acatagcccacaccgttctc

SEQ ID NO: 9 - DNA, 20 bp, Artificial Sequence
  PRIMER
cgggaagaagatggtgctgt

SEQ ID NO: 10 - DNA, 18 bp, Artificial Sequence
  PRIMER
ttggacggggctggttat

SEQ ID NO: 11 - DNA, 19 bp, Artificial Sequence
  PRIMER
ccacacgaggcgtgaactc

SEQ ID NO: 12 - DNA, 21 bp, Artificial Sequence
  PRIMER
cttcaggttacatcggggtga

SEQ ID NO: 13 - DNA, 19 bp, Artificial Sequence
  PRIMER
cctagcctcatacccccag

SEQ ID NO: 14 - DNA, 19 bp, Artificial Sequence
  PRIMER
caaaccgccaggcagattg

SEQ ID NO: 15 - DNA, 20 bp, Artificial Sequence
  PRIMER
cagcagaggtgtgtactatg

SEQ ID NO: 16 - DNA, 20 bp, Artificial Sequence
  PRIMER
gcgttgttcttacttcgagc

SEQ ID NO: 17 - DNA, 24 bp, Artificial Sequence
  PRIMER
ctgtccagaccaagggtacagcct

SEQ ID NO: 18 - DNA, 24 bp, Artificial Sequence
  PRIMER
gaggtatagtgggacacatagtgg

SEQ ID NO: 19 - DNA, 23 bp, Artificial Sequence
  PRIMER
cctcacattcttaggactggagc

SEQ ID NO: 20 - DNA, 19 bp, Artificial Sequence
  PRIMER
ggcaatcagagctctcgga

SEQ ID NO: 21 - DNA, 20 bp, Artificial Sequence
  PRIMER
cctttgacaagcggactctc

SEQ ID NO: 22 - DNA, 20 bp, Artificial Sequence
  PRIMER
gccagcataaaaacccttca

SEQ ID NO: 23 - DNA, 20 bp, Artificial Sequence
  PRIMER
ccatgatgttctcgggtttc

SEQ ID NO: 24 - DNA, 20 bp, Artificial Sequence
  PRIMER
tgtcaccgtggggataaagt

SEQ ID NO: 25 - DNA, 20 bp, Artificial Sequence
  PRIMER
atgtgcacgaaaatggaaca

SEQ ID NO: 26 - DNA, 20 bp, Artificial Sequence
  PRIMER
cctgacccgaaaagtagctg

SEQ ID NO: 27 - DNA, 19 bp, Artificial Sequence
  PRIMER
cgctgacatccgcaacatt

SEQ ID NO: 28 - DNA, 19 bp, Artificial Sequence
  PRIMER
ggcctcttgcccagtttca

SEQ ID NO: 29 - DNA, 22 bp, Artificial Sequence
  PRIMER
gaccaccttggcaatgtctctg

SEQ ID NO: 30 - DNA, 24 bp, Artificial Sequence
  PRIMER
tggctgaggaagtcatctgagttg

SEQ ID NO: 31 - DNA, 22 bp, Artificial Sequence
  PRIMER
gcatcacagggaagaccgtgtc

SEQ ID NO: 32 - DNA, 25 bp, Artificial Sequence
  PRIMER
gaagttcaatgtcggagtttctgag

SEQ ID NO: 33 - DNA, 22 bp, Artificial Sequence
  PRIMER
tggtgccaaccactatccatgc

SEQ ID NO: 34 - DNA, 20 bp, Artificial Sequence
  PRIMER
cacaagtgccgccatgacag

SEQ ID NO: 35 - DNA, 24 bp, Artificial Sequence
  PRIMER
tgaggcttctcttggtgtccatac

SEQ ID NO: 36 - DNA, 21 bp, Artificial Sequence
  PRIMER
aaagggtgtcattactgcggg

SEQ ID NO: 37 - DNA, 16 bp, Artificial Sequence
  PRIMER
tggggggcaactcggc

SEQ ID NO: 38 - DNA, 18 bp, Artificial Sequence
  PRIMER
ggaatgatctaagcccag

SEQ ID NO: 39 - DNA, 23 bp, Artificial Sequence
  PRIMER
gctgtaacaaatgtgaaccagga

SEQ ID NO: 40 - DNA, 21 bp, Artificial Sequence
  PRIMER
gccttgcctgtatcacaaact

SEQ ID NO: 41 - DNA, 20 bp, Artificial Sequence
  PRIMER
ggacttcgagcaagagatgg

SEQ ID NO: 42 - DNA, 22 bp, Artificial Sequence
  PRIMER
tgtgttggggtacaggtctttg

(19) United States
(12) Patent Application Publication
(10) Pub. No.: US 2016/0220567 A1
(43) Pub. Date: Aug. 4, 2016

(54) COMPOSITION FOR TREATING OR PREVENTING BONE DISEASES, COMPRISING HALOFUGINONE AS ACTIVE INGREDIENT

(71) Applicant: THE CATHOLIC UNIVERSITY OF KOREA INDUSTRY-ACADEMIC COOPERATION FOUNDATION, Seoul (KR)

(72) Inventors: Sung-Hwan Park, Seoul (KR); Mi-La Cho, Seoul (KR); MI-Kyung Park, Gyeonggi-do (KR); Seong-Ki Kwok, Seoul (KR); Jennifer Lee, Seoul (KR); Jin-Sil Park, Seoul (KR); Sung-Min Kim, Seoul (KR); Mi-Ae Lim, Seoul (KR); Seong-Ye Baek, Seoul (KR); Dong-Gun Lee, Incheon (KR); Eun-Mi Park, Gyeonggi-do (KR)

(21) Appl. No.: 14/917,548

(22) PCT Filed: Sep. 5, 2014
(86) PCT No.: PCT/KR2014/008426

§ 371 Date: Mar. 8, 2016

(30) Foreign Application Priority Data
Sep. 9, 2013 (KR) 10-2013-0107821
Sep. 4, 2014 (KR) 10-2014-0117469

(51) Int. Cl. A61K 31/517

(52) U.S. Cl. CPC A61K 31/517

(57) ABSTRACT

The present invention relates to a composition for treating or preventing bone diseases comprising halofuginone as an active ingredient and, more specifically, to a composition for treating or preventing bone diseases, comprising, as an active ingredient, halofuginone having an osteoclast differentiation inhibitory efficacy and an osteoblast differentiation stimulatory activity. The composition for treating or preventing bone diseases, comprising halofuginone as an active ingredient, according to the present invention, reduces the expression of Th17 cells and increases the expression of Treg cells, thereby representing an effect of inhibiting osteoclast differentiation and being capable of also effectively inhibiting bone resorption by osteoclasts. Furthermore, the composition of the present invention will be able to be usefully utilized as a use for the treatment or prevention of bone diseases by being able to promote the differentiation and activity of osteoblast.

### TECHNICAL FIELD

The present invention relates to a composition for treating or preventing bone diseases containing halofuginone as an active ingredient, and more specifically, to a composition for treating or preventing bone diseases containing as an active ingredient, halofuginone, which has the effects of inhibiting osteoclast differentiation and activating osteoblast-related factors.

### BACKGROUND ART

The bones constituting our body undergo a life-long process called bone remodeling, through which old bones are replaced with new bones, as in the case with normal body tissues. The process progresses while a balance is maintained between osteoblasts, which cause bone formation, and osteoclasts, which cause bone destruction. During the adolescence years, bone formation occurs more actively than bone destruction, and, in adults, a normal bone remodeling occurs with a balance between bone formation and bone destruction. However, in elderly people, bone destruction occurs more actively than bone formation thus causing a disease. When there is an imbalance during the bone remodeling, as such, it may lead to bone diseases, such as rheumatoid arthritis; osteoporosis, which occurs due to a decrease in bone density by osteoclasts; osteopetrosis, which occurs due to an increase in bone density by excessively active osteoblast activity; and Paget's disease called osteitis deformans.

An osteoclast is a large multinucleated cell which destroys or absorbs bone tissue that has become unnecessary during the process of bone growth in vertebrate animals. When the activity of osteoclast becomes too active it results in a disease such as osteoporosis due to low bone density. The differentiation into osteoclast requires receptor activator of nuclear factor-kB ligand (RANKL), whereas the proliferation of osteoclast precursor requires macrophage colony stimulating factor (M-CSF). The differentiation of osteoclast begins when RANKL binds to its receptor, RANK. When RANKL binds to RANK, the adaptor molecules such as tumor necrosis factor receptor associated factor 6 (TRAF6) become activated. TRAF2, TRAF5, and TRAF6, which are known as TRAF family members, activate transcription factors such as NF-kB and activator protein-1 (AP-1), which are necessary for the differentiation of osteoclast. When TRAF6 is activated by the RANKL signal for the differentiation of osteoclast, PI3K, transforming growth factor β-activated kinase (TAK1), Akt/PKB, and MAPKs, which include JNK, ERK, and p38, and in addition, NF-kB, c-Fos, Fra-1, cyclic-AMP-responsive-element-binding protein (CREB), and nuclear factor of activated T cells cytoplasmic 1 (NFATc1) are activated. These inductions can occur by the c-Fos pathway through the signals of TRAF6-NF-kB and RANKL-RANK. Furthermore, osteoclast-specific genes, such as cathepsin K, tartrate-resistant acid phosphatase (TRAP), calcitonin receptor and osteoclast-associated receptor (OSCAR) are expressed by them. The process of attachment of osteoclast to bone and subsequent breakdown of bone induces cytoskeleton reorganization, which includes the formation of a sealing zone, which is formed when osteoclast attaches to bone surface around the bone erosion space, and filamentous actin ring. The RANKL/RANK/TRAF6 axis and NFATc1 are essential for the differentiation into mature osteoclast, and these signaling pathways are initiated by RANKL, and the molecules associated therein may become targets of drugs for bone diseases. However, the RANKL/RANK system and the protein kinases and transcription factors in the system are involved in biological processes such as immune system as well as in the formation of osteoclast, and thus these factors may exhibit serious adverse effects when they become targets for therapeutic agents, thus requiring careful approaches.

Additionally, osteoblasts are formed after being derived from mesenchymal stem cells, and the mineralization including calcium, etc., being formed by the differentiation of osteoblasts, can not only maintain bone strength but also play a very important role in homeostasis of the calcium in the entire body and hormone metabolism. The calcium formation by the differentiation of osteoblasts is controlled by vitamin D, parathyroid hormone, etc., and alkaline phosphatase (ALP), which is associated with the differentiation of osteoblasts, is synthesized in cells at the initial differentiation stage by the cross-talk of various signaling system, such as bone morphogenetic protein (BMP), Wnt, MAP kinase, calcineurin-calmodulin kinase, NF-κB, AP-1, etc., and osteopontin, osteocalcin, type 1 collagen, etc., which are associated with mineralization are synthesized, thereby resulting in bone formation by differentiation of osteoblasts.

Meanwhile, the bones in humans should maintain homeostasis through the processes of formation and remodeling, and the remodeling occurs in the bone multicellular unit (BMU) consisting of osteoblasts and osteoclasts, and these cells are known to play an important role in the respective process of bone formation and bone resorption. When there is damage in the cooperation system between these two different kinds of cells during the processes, various bone diseases including osteoporosis may occur.

Accordingly, there is recently a growing effort to effectively produce the osteoblasts and strengthen or remedy the functions of bone tissue, and ascorbic acid, β-glycerophosphate, dexamethasone, etc., which are currently used as osteogenic supplements, have been reported to be effective in inducing the differentiation of osteoblasts and transforming growth factor-beta (TGF-beta) subfamily, which includes activin, bone morphogenic protein (BMP), inhibin, growth/differentiation factor (GDF), etc., has been known to play an important role in formation and maintenance of bone tissue. However, excellent therapeutic agents without adverse effects have not been available.

As the representative disease among the various diseases in bone metabolism, osteoporosis is a disease characterized by a decrease in bone mass and deterioration of microstructures of bone tissue thus causing a continuous increase in the risk of bone fracture, in which minerals (in particular, calcium) and substrates which constitute bones are in a decreased state. Osteoporosis occurs in a state when the balance in bone remodeling is lost and thus osteoclast action is increased compared to osteoblast action. The interior of the normal bone structure provides a dense structure like a mesh. However, in the case of osteoporosis, the space between bone structures becomes wider and the microstructure becomes thin and weakened thus being progressed into a state that bones can be easily fractured even with a small shock. Osteoporosis can be classified into: a postmenopausal osteoporosis, in which rapid bone loss (annual loss of 2% to 3%) occurs simultaneously with the start of menopause and thus there is an increase in pressure on the spine and risk of easy fracture of carpals; senile osteoporosis, which gradually occurs in old men and women aged 70 or higher (0.5% to 1% per year) and brings a progressive bone loss in hip bone and spine; and a secondary osteoporosis, which occurs, regardless of ages, due to diseases (endocrine disease, gastrointestinal disease, and malignant tumor), drugs (adrenocortical hormone, chemotherapy, thyroid hormone, anticonvulsants, anticoagulants, methotrexate, cyclosporine, GnRH, etc.), alcohols, smoking, accidents, etc.

It has been known that bone metastasis almost always occurs in patients with breast cancer, prostate cancer, or multiple myeloma and the residual life expectancies of these cancer patients are influenced by the presence of bone metastasis. The relatively high death rate of patients with breast cancer or prostate cancer is ascribed to the selective metastasis of cancer cells into bones. The bone metastasis observed in breast cancer is mostly an osteolytic metastasis and it is known that breast cancer cells do not directly affect the bones but stimulate osteoclasts to cause the osteolytic metastasis. Meanwhile, bone metastasis observed in prostate cancer is an osteoblastic metastasis. The osteoblastic metastasis is also known to be closely associated with osteolysis. The cancer cells metastasized into bones proliferate in a microenvironment around bones and stimulate the activity of osteoclasts or osteoblasts and determine whether they will be progressed into the osteolytic metastasis or the osteoblastic metastasis. Bone metastasis occurs in about 80% of breast cancer patients and the metastatic breast cancer cells activate osteoclasts. The activated osteoclasts destroy the balance in the microenvironment around the bones to cause osteolysis, which then results in frequent pathologic fracture and also bone-related diseases such as leukoerythroblastic anemia, bone deformities, hypercalcemia, pains, and nerve compression syndromes.

The therapeutic agents currently used for the treatment of osteoporosis and bone damage caused by bone metastasis of cancer cells are bisphosphonate agents such as Fosamax® (component: alendronate) and Actonel® (component: risedronate). Most of these bisphosphonate agents serve to weaken the functions of osteoclasts, which destroy bones, and induce apoptosis thereby slowing or stopping the loss of bones. However, recently, growing incidents of osteonecrosis of jaw bone, severe atrial fibrillation, deterioration of bones or joints or musculoskeletal pains in patients administering bisphosphonate agents. Accordingly, main interest has been focused on the development of a therapeutic agent for the prevention and treatment of osteoporosis, capable of reducing side effects and effectively inhibiting bone resorption.

### DISCLOSURE

### Technical Problem

The present inventors, while endeavoring to develop a therapeutic agent for effectively treating or preventing bone diseases, have discovered that halofuginone is very excellent for the treatment or prevention of bone diseases, thereby completing the present invention.

Accordingly, an object of the present invention is to provide a pharmaceutical composition for treating or preventing bone diseases, containing a halofuginone compound or a salt thereof as an active ingredient.

Another object of the present invention is to provide a composition for promoting the differentiation or activation of osteoblasts, containing a halofuginone compound or a salt thereof as an active ingredient.

Still another object of the present invention is to provide a composition for inhibiting the differentiation of osteoclasts, containing a halofuginone compound or a salt thereof as an active ingredient.

Still another object of the present invention is to provide a food composition for improving bone functions, containing a halofuginone compound or a salt thereof as an active ingredient.

Still another object of the present invention is to provide a method of treating or preventing bone diseases including administering an effective amount of a halofuginone compound or a salt thereof to a subject.

Still another object of the present invention is to provide a method for promoting the differentiation or activity of osteoblasts, the method comprising administering an effective amount of a halofuginone compound or a salt thereof to a subject.

Still another object of the present invention is to provide a method for inhibiting osteoclast differentiation, the method comprising administering an effective amount of a halofuginone compound or a salt thereof to a subject.

### Technical Solution

Accordingly, the present invention provides a pharmaceutical composition for treating or preventing bone diseases, containing a halofuginone compound or a salt thereof as an active ingredient.

In an exemplary embodiment of the present invention, the bone disease may be any one selected from the group consisting of osteoporosis, osteoarthritis, osteopetrosis, Paget's disease, osteomalacia, rickets, ossifying fibroma, adynamic bone diseases, metabolic bone diseases, and rheumatoid arthritis, which is a bone-destroying disease through bone damage caused by bone metastasis of cancer cells and immune inflammatory response.

In an exemplary embodiment of the present invention, the halofuginone may exhibit a therapeutic effect by reducing or inhibiting the differentiation of osteoclasts.

In an exemplary embodiment of the present invention, the halofuginone may exhibit a therapeutic effect by reducing or inhibiting osteoclast differentiation induced by RANKL.

In an exemplary embodiment of the present invention, the halofuginone may reduce or inhibit osteoclast differentiation by the inhibition of Th17 expression.

In an exemplary embodiment of the present invention, the halofuginone may inhibit the expression of Th17 cells by activating ERK in the Th17 cells.

In an exemplary embodiment of the present invention, the halofuginone may exhibit a therapeutic effect by inhibiting the bone resorption by osteoclasts.

In an exemplary embodiment of the present invention, the halofuginone may promote the differentiation or development of osteoblasts.

In an exemplary embodiment of the present invention, the halofuginone may be administered to a subject in an amount of from 0.5 μg/kg to 2000 μg/kg.

Additionally, the present invention provides a composition for promoting the differentiation or activity of osteoblasts, containing a halofuginone compound or a salt thereof as an active ingredient.

In an exemplary embodiment of the present invention, the halofuginone may promote the expression or activity of SOX6 or Rcan3, which are factors associated with osteoblast differentiation.

Additionally, the present invention provides a composition for inhibiting osteoclast differentiation, containing a halofuginone compound or a salt thereof as an active ingredient.

Additionally, the present invention provides a food composition for improving bone functions, containing a halofuginone compound or a salt thereof as an active ingredient.

Additionally, the present invention provides a method of treating or preventing bone diseases including administering an effective amount of a halofuginone compound or a salt thereof to a subject.

Hereinafter, the present invention will be described in more details.

The present invention is characterized in providing a pharmaceutical composition for treating or preventing bone diseases containing halofuginone as an active ingredient.

The present inventors, while studying to develop a novel therapeutic agent for treating bone diseases, have confirmed that halofuginone can be useful as a novel therapeutic agent for treating bone diseases.

Halofuginone, which is an active ingredient contained in the composition of the present invention, is a compound having the following Formula 1, and is a coccidiostat used in the veterinary field. Additionally, halofuginone is a synthetic halogenated derivative of febrifuginine, which is a natural alkaloid quinazolinone discovered in a Chinese plant, Dichroa febrifuga. Collgard Biopharmaceuticals Ltd. has been developing halofuginone for the treatment of scleroderma, and it has been granted as an orphan drug by the US FDA. Additionally, halofuginone is known to inhibit the growth of tumor cells by inhibiting the expression of collagen type I gene, and acts as a high-affinity inhibitor against glutamyl prolyl tRNA synthetase, and has also been known to act as a signal for the initiation of amino acid deficiency response by inhibiting the amino acid charging by prolyl tRNA.

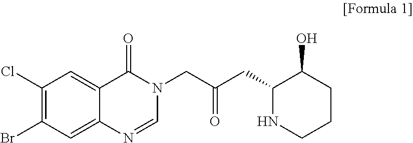 [SMILES: O=C(C[C@H]1NCCC[C@@H]1O)Cn1cnc2cc(Br)c(Cl)cc2c1=O]

However, there has been no report regarding the use of halofuginone as a therapeutic agent for the treatment of bone diseases.

Examples of the pharmacological efficacies confirmed by exemplary embodiments of the present invention include the inhibition of bone resorption by inhibiting the differentiation of osteoclasts induced by receptor activator of NF-[kappa] B ligand (RANKL), and the reduction or inhibition of the differentiation of osteoclasts by inhibiting the expression of Th17, and in particular, the inhibition of the expression of Th17 by activating the ERK in Th17 cells.

Accordingly, the present invention can provide a pharmaceutical composition for treating or preventing bone diseases, containing a halofuginone compound or a salt thereof as an active ingredient.

The halofuginone used in the present invention may be synthesized by a chemical synthesis or may be isolated from a natural product and then purified for use.

Additionally, the halofuginone used in the present invention may be preferably in the form of a pharmaceutically acceptable salt. The salt may be an acid addition salt formed by a pharmaceutically acceptable free acid and for the free acid, organic acids or inorganic acids may be used. The organic acids may include citric acid, acetic acid, lactic acid, tartaric acid, maleic acid, fumaric acid, formic acid, propionic acid, oxalic acid, trifluoroacetic acid, benzoic acid, gluconic acid, methanesulfonic acid, glycolic acid, succinic acid, 4-toluenesulfonic acid, glutamic acid, and aspartic acid, although not limited thereto. Additionally, the inorganic acids may include hydrochloric acid, bromic acid, sulfuric acid, and phosphoric acid, although not limited thereto.

Preferably, the halofuginone compound or a pharmaceutically acceptable salt thereof, being an amount that can inhibit the differentiation of osteoclasts or promote the differentiation or activity of osteoblasts, may be administered to a subject in an amount of from 0.5 μg/kg to 2000 μg/kg, and preferably, may be administered to a subject in an amount of from 8 μg/kg to 40 μg/kg.

In particular, in an exemplary embodiment of the present invention, it was confirmed that the effective amount of the halofuginone compound that can draw out pharmacological efficacies, i.e., the inhibitory activity against the differentiation of osteoclasts or the promoting activity of the differentiation or activity of osteoblasts is 100 μg/kg to 500 μg/kg, and the concentration of a pharmacological material for treatment confirmed by the animal experiment can be used to estimate the applicable concentration of the pharmacological material for treatment in humans using the equation known in the art shown below.

Concentration for application in humans (mg/kg)=[Concentration for application in animals (mg/kg) [symbol] Km index score for application in animals]/[Km index score for application in humans]

In particular, Km index score refers to a determined converted into a body surface area relative to the body of each individual, and it is determined to be 37 for human adults, 25 for human children, 3 for mice, and 6 for rats.

Accordingly, according to the calculation based on the above equation, the concentration of the halofuginone for application in humans (adults), converted based on the concentration administered to the animal model, mice, was confirmed to be in an amount of from 8 μg/kg to 40 μg/kg.

As used herein, the term “a pharmaceutically acceptable salt” refers to a salt prepared by a conventional method and may be used by a method known in the art without limitation. “A pharmaceutically acceptable salt” includes the salts derived from the following pharmacological or physiologically acceptable inorganic acids and organic acids and bases but are not limited thereto. Examples of the appropriate acids may include hydrochloric acid, bromic acid, sulfuric acid, nitric acid, perchloric acid, fumaric acid, maleic acid, phosphoric acid, glycolic acid, lactic acid, salicylic acid, succinic acid, toluene-p-sulfonic acid, tartaric acid, acetic acid, citric acid, methanesulfonic acid, formic acid, benzoic acid, malonic acid, naphthalene-2-sulfonic acid, benzenesulfonic acid, etc. The salt derived from appropriate bases may include, for example, sodium, alkali earth metal, e.g., magnesium, ammonium, etc.

The halofuginone or a salt thereof of the present invention may further include an appropriate carrier, excipient, or diluent, according to the conventional method.

As used herein, the term “carrier” refers to a carrier, excipient, or diluent which neither significantly stimulate organism nor inhibit the biological activity and characteristics of the compound to be administered.

Examples of the carrier, excipient, and diluent to be included in the composition of the present invention may include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, mineral oil, etc.

The composition according to the present invention containing halofuginone or a salt thereof may be prepared in the form of an oral formulation including powders, granules, tablets, capsules, suspensions, emulsions, syrups, and aerosols; formulations for external use; suppositories; and sterile injection solutions, according to the conventional method.

Specifically, the formulations may be prepared using the commonly used diluents or excipients such as fillers, extenders, binders, humectants, disintegrants, and surfactants. Solid preparations for oral administration may include tablets, pills, powders, granules, capsules, etc. These solid preparations may be prepared by adding at least one excipient, e.g., starch, calcium carbonate, sucrose, lactose, gelatin, etc., to the extract. Additionally, lubricants such as magnesium stearate, talc, etc., may be used in addition to the simple excipients. Examples of liquid formulations for oral administration may include suspensions, preparations for internal use, emulsions, syrups, etc., and various kinds of excipients, e.g., humectants, sweeteners, fragrances, preservatives, etc., may be used in addition to the commonly used simple diluents such as water and liquid paraffin. Formulations for parenteral administration may include sterile aqueous solutions, non-aqueous solvents, suspensions, emulsions, lyophilized preparations, and suppositories. Examples of non-aqueous solvents, suspensions may include propylene glycol, polyethylene glycol, a vegetable oil such as olive oil, an injectable ester such as ethylolate, etc. Examples of suppository bases include Witepsol, Macrogol, Tween 61, cacao butter, laurinum, glycerogelatin, etc.

As used herein, the term “osteoclast” refers to a cell which appears during the formation or regeneration of bones, and there occurs bone formation at one end of bone tissue while bone destruction or bone resorption occurs at the other end of bone tissue. As disclosed in the prior art above, in general, the two major types of cells, bone-forming osteoblasts and bone-destroying osteoclasts, are involved in bone formation, and the close association between the two types of cells enables to maintain the homeostasis of bones. For example, osteoblasts help to maintain the bone homeostasis in the body by controlling the differentiation of osteoclasts which are in charge of bone resorption via release of materials, such as receptor activator of nuclear factor-κB ligand (RANKL) and its decoy receptor, osteoprotegerin (OPG), that is, upon binding to the RNAK, which is the surface receptor of the osteoclast precursor cells, the osteoclast precursor cells are matured into osteoclasts and thereby bone resorption occurs, whereas the binding of OPG to RANKL blocks the binding between RANKLand RANK thereby inhibiting the formation of osteoclasts. Accordingly, the osteoclasts being formed from precursor cells are involved in resorption or destruction of old bones, and maintenance of body functions by releasing a small amount of calcium accumulated in bones into the blood stream. However, excessively activated osteoclasts destroy the balance in the microenvironment around the bone to be subjected to osteolysis thereby causing bone-related diseases such as pathological fracture, leukopenia, bone deformities, hypercalcemia, pains, and nerve compression syndromes.

Accordingly, the excessive differentiation and activity of osteoclasts may become a cause of bone diseases.

Additionally, as used herein, the term “osteoblast” refers to a cubic type cell for bone formation, which is involved in bone formation and bone remodeling. Osteoblasts change the collagen into a lattice shape in the part decomposed by osteoclasts, and attach calcium, magnesium, phosphorous, etc., therein to renew bone matrices thereby ultimately playing a role of bone formation.

Furthermore, in the present invention, the effect of halofuginone on the expression of SOX6 and Rcan3 genes was analyzed in order to confirm whether halofuginone is involved in the differentiation or development of osteoblasts. As a result, it was confirmed that halofuginone plays a role of promoting expression or activity of the genes associated with the differentiation or development of osteoblasts.

The Regulators of calcineurin (Rcan3) is known as a calcineurin inhibitor which can promote bone formation by reducing the expression of NFAT downstream genes and Sox6 is known as a factor that can promote the functions of chondroblasts.

In this regard, it was confirmed that the composition of the present invention can play a role of increasing the expression of the genes associated with the differentiation or development of osteoblasts, thereby ultimately contributing to the bone formation through the promotion of development of osteoblasts.

Accordingly, the present invention not only provides a pharmaceutical composition for treating or preventing bone diseases, containing a halofuginone or a salt thereof as an active ingredient, but also provides a composition capable of promoting the differentiation or development of osteoblasts containing a halofuginone or a salt thereof as an active ingredient, and a composition capable of inhibiting the differentiation of osteoclasts.

As used herein, the term “metabolic bone diseases” refers to disorders that occur as a result of problems in bone formation by the lack of osteoblasts or the differentiation of osteoclasts and may include osteoporosis, osteoarthritis, osteopetrosis, Paget's disease, osteomalacia, rickets, ossifying fibroma, adynamic bone diseases, metabolic bone diseases, and rheumatoid arthritis, which is a bone-destroying disease through bone damage caused by bone metastasis of cancer cells and immune inflammatory response, although not limited thereto.

Additionally, the present invention provides a method for treating or preventing the bone diseases, which includes administering an effective amount of a halofuginone compound or a salt thereof to a subject.

As used herein, the term “prevention” refers to all actions associated with the improvement or advantageous changes in symptoms of the bone diseases by administering the composition containing halofuginone or a pharmaceutically acceptable salt thereof according to the present invention.

As used herein, the term “treatment” refers to all actions associated with the inhibition or delay of the occurrence of the bone diseases by administering the composition containing halofuginone or a pharmaceutically acceptable salt thereof according to the present invention.

As used herein, the term “administration” refers to an introduction of the composition of the present invention to a patient by an appropriate method, and the composition of the present invention may be administered via various oral or parenteral routes as long as the composition can arrive at a target tissue. Specifically, the composition of the present invention may be administered via oral-, rectal-, topical-, intravenous-, intraperitoneal-, intramuscular-, intraarterial-, dermal-, intranasal-, inhaled-, intraocular-, or intradermal administration. The method of treatment according to the present invention may include administering an effective amount of the composition of the present invention. It should be obvious to one of ordinary skill in the art that an appropriate total daily dose can be determined by the practitioner within the scope of the correct medical decision. Preferably, the specific therapeutically effective dose for a particular patient should be applied differently according to various factors and similar factors well known in the medical field, including the kinds and degrees of responses to be achieved; specific composition(s) including whether a different agent is used depending on cases; age, weight, general health conditions, sex and diet of the patient, administration time, administration route and release rate of the composition, period of treatment; and drugs to be administered in combination or simultaneously with the specific composition. Accordingly, the effective dose of the composition suitable for the purpose of the present invention may be preferably determined considering the factors described above.

Furthermore, the present invention provides a method for treating or preventing metabolic bone diseases including administering a composition containing halofuginone or a pharmaceutically acceptable salt thereof to a subject to be needed.

The method of the present invention for treating or preventing metabolic bone diseases may be applicable to any subject in which any of the metabolic bone diseases has occurred, and the animals to be administered may include domestic animals, such as, cattle, pigs, sheep, horses, dogs, and cats, in addition to humans and primates.

Additionally, the present invention can also provide a food composition for improving bone functions containing halofuginone or a pharmaceutically acceptable salt thereof as an active ingredient.

The composition of the present invention containing halofuginone or a pharmaceutically acceptable salt thereof can be variously used including pharmaceutical drugs, foods, and beverages for preventing bone metabolism-related diseases. The foods, in which the extract of the present invention can be added, may include various kinds of foods, e.g., beverages, gums, teas, vitamin complexes, health supplementary foods, etc., and may be used in the form of pills, powders, granules, infusions, tablets, capsules, or beverages.

In particular, the amount of the halofuginone contained in the foods or beverages may be in general, in the case of the health food composition of the present invention, in an amount of from 0.01 wt % to 15 wt % relative to the weight of the total food, and in the case of the health beverages, in an amount of 0.02 g to 10 g relative to 100 mL of the total beverage, preferably 0.3 g to 1 g.

The health beverage composition of the present invention is not particularly limited regarding the liquid ingredients except that halofuginone is contained as an essential ingredient at an indicated ratio, and various kinds of flavoring agents or natural carbohydrates may be further contained as additional ingredients, as in the case of the conventional beverages. Examples of the natural carbohydrates may include conventional saccharides such as monosaccharides (e.g., glucose, fructose, etc.), disaccharides (e.g., maltose, sucrose, etc.), and polysaccharides (e.g., dextrin, cyclodextrin, etc.); and sugar alcohol such as xylitol, sorbitol, erythritol, etc. As flavoring agents, in addition to those described above, natural flavoring agents (thaumatin, stevia extract (e.g., rebaudioside A, glycyrrhizin, etc.) and synthetic flavoring agents (saccharin, aspartame, etc.) may be used advantageously. The ratio of the natural carbohydrate may be generally about 1 g to 20 g per 100 mL of the composition of the present invention, and preferably, about 5 g to 12 g.

The composition of the present invention may further contain various nutrients, vitamins, minerals (electrolytes), flavoring agents such as synthetic flavoring agents and natural flavoring agents, coloring agents and enhancers cheese, chocolate, etc.), pectic acid and a salt thereof, alginic acid and a salt thereof, organic acids, protective colloid thickeners, pH adjusters, stabilizers, preservatives, glycerins, alcohols, carbonating agents used for carbonated beverages, etc. The compositions of the present may further contain natural fruit juice and fruit pulp for preparation of fruit juices or vegetable juices. These ingredients may be used alone or in combination. The content of the additives is not so significant but may be selected within the range of 0 parts to about 20 parts by weight per 100 parts by weight of the composition of the present invention

The food composition of the present invention is not particularly limited regarding the ingredients except that the composition contains halofuginone or a pharmaceutically acceptable salt thereof having an effect on bone diseases as an essential ingredient, and various kinds of herbal drug extracts, food supplementary additives or natural carbohydrates may be further contained as additional ingredients, as in the case of the conventional foods.

Additionally, as described above, food supplementary additives may be further contained, and examples of the food supplementary additives may include the conventional food supplementary additives known in the art, e.g., flavoring agents, coloring agents, fillers, stabilizers, etc.

Examples of the natural carbohydrates may include conventional saccharides such as monosaccharides (e.g., glucose, fructose, etc.), disaccharides (e.g., maltose, sucrose, etc.), and polysaccharides (e.g., dextrin, cyclodextrin, etc.); and sugar alcohol such as xylitol, sorbitol, erythritol, etc. As flavoring agents, in addition to those described above, natural flavoring agents (thaumatin, stevia extract (e.g., rebaudioside A, glycyrrhizin, etc.) and synthetic flavoring agents (saccharin, aspartame, etc.) may be used advantageously.

The food composition of the present invention may further contain various nutrients, vitamins, minerals (electrolytes), flavoring agents such as synthetic flavoring agents and natural flavoring agents, coloring agents and enhancers (e.g., cheese, chocolate,eta:.), pectic acid and a salt thereof, alginic, acida la salt thereof, organic acids, protective colloid thickeners, pH adjusters, stabilizers, preservatives, glycerins, alcohols, carbonating agents used for carbonated beverages, etc. The composition of the present invention may further contain natural fruit juice and fruit pulp for preparation of fruit juices or vegetable juices. These ingredients may be used alone or in combination.

### Advantageous Effects

The composition of the present invention for treating or preventing bone diseases containing halofuginone as an active ingredient has the effect of inhibiting osteoclast differentiation by reducing the expression of Th17 cells while increasing the expression of Treg cells and also the effect of effectively inhibiting the bone resorption by osteoclasts, thus being expected to be effectively used for the treatment or prevention of bone diseases.

### DESCRIPTION OF DRAWINGS

FIGS. 1A to 1D illustrate the effect of halofuginone on treating arthritis analyzed via evaluation of arthritis index score (FIG. 1A), joint tissue staining (FIG. 1B), intraarticular inflammatory cytokine staining (FIG. 1C), and the amount of IgG, IgG1, and IgG2a in sera of each animal group (FIG. 1D), after administration of halofuginone in an animal model with arthritis.

FIGS. 2A to 2C illustrate the expression of Treg cells and Th17 cells in the spleen tissues of each group (FIG. 2A), the expression of pSTAT3 and pSTAT5 (FIG. 2B), and the expression of IL-17 and Foxp3 in spleen cells or lymph node cells of each group (FIG. 2C), observed after administration of halofuginone in an animal model with arthritis.

FIGS. 3A to 3D illustrate the differentiation of Th17 cells (FIG. 3A), the expression of IL-17, CCR6, SOCS3, and Foxp3 in Th17 cells (FIG. 3B), the expression of pERK (FIG. 3C), and the amount of IL-17 production in T cells via the IDO in dendritic cells and the increase of HF-mediated IDO expression in dendritic cells (FIG. 3D), observed in T cells treated with halofuginone.

FIGS. 4A to 4C illustrate the TRAP staining in joints (FIG. 4A), the TRAP staining of bone marrow cells after differentiation into osteoclasts (FIG. 4B), and the mRNA expression of factors associated with osteoclast differentiation in osteoclasts of the differentiated osteoclasts of FIG. 4B (FIG. 4C), observed after administration of halofuginone in an animal model with arthritis.

FIGS. 5A to 5D illustrate the effects of halofuginone on the expression of TRAP (FIG. 5A), the degree of bone resorption (FIG. 5B), the expression of c-Fos, JunB, and Jdp2 in osteoclasts (FIG. 5C), and the cell cycle and the expression of ccdn1 (FIG. 5D), observed during the process of osteoclast differentiation.

FIGS. 6A to 6D illustrate the effects of halofuginone observed on the differentiation of Th17 cells and Treg cells during the process of human T cell differentiation (FIG. 6A), the expression of TRAP during the process of human osteoclast differentiation (FIG. 6B), the expression of NFATc1, CTR, cathepsin K, and RANK in the cells of FIG. 6B (FIG. 6C), and the bone resorption (FIG. 6D).

FIG. 7 illustrates the expression levels of SOX6 and Rcan3, which are factors associated with the differentiation and development of osteoblasts, analyzed via immunohistochemical staining method for a group of DBA/1J mice treated with halofuginone (500 μg/kg) and a group of DBA/1J mice not treated with halofuginone (500 μg/kg), after immunization of the DBA/1J mice with type II collagen.

### MODES OF THE INVENTION

Hereinafter, exemplary embodiments of the present invention will be described in detail. However, the present invention is not limited to the exemplary embodiments disclosed below, but can be implemented in various forms. The following exemplary embodiments are described in order to enable those of ordinary skill in the art to embody and practice the invention.

### Example 1

### Analysis of the Therapeutic Effect of a Halofuginone Compound on Arthritis

<1-1> Injection of a Halofuginone Compound to an Animal Model with Arthritis

For the construction of a mouse model with collagen-induced arthritis (hereinafter, referred to as “CIA”), a 6-week old male DBA/1J mouse was subjected to primary immunization by intradermal injection after mixing the bovine type II collagen (hereinafter, referred to as “CII”) and complete Freud's adjuvant (CFA) in an amount of 100 μL, respectively. After a week thereafter, halofuginone was injected intraperitoneally at 3 day intervals at a concentration of 100 μg/kg and 500 μg/kg, respectively (9 weeks).

### <1-2> Measurement of Arthritis Index Score

To examine the therapeutic effect of treating halofuginone of the present invention on arthritis, the clinical score of the joint inflammation of the mouse prepared in Example <1-1> (the mouse with arthritis injected with halofuginone) was measured while observing the mouse. The group treated with a solution in which DMSO was diluted in saline was used as the control group.

The clinical score was evaluated using a clinical score system, in which the respective score for swelling and redness on toes or caudal regions was determined by observing daily the degree of the progress of the joint inflammation on the arthritis lesion of the mouse by the naked eye.

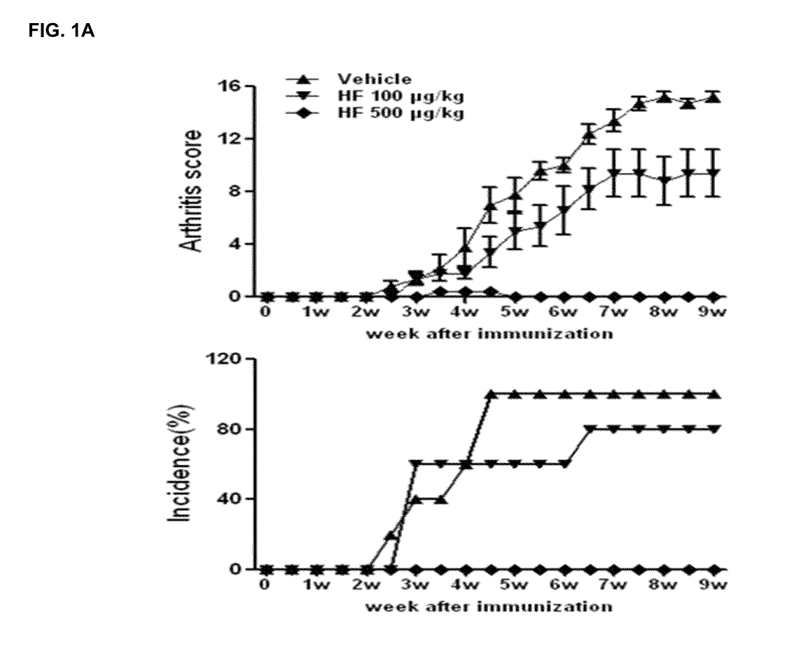

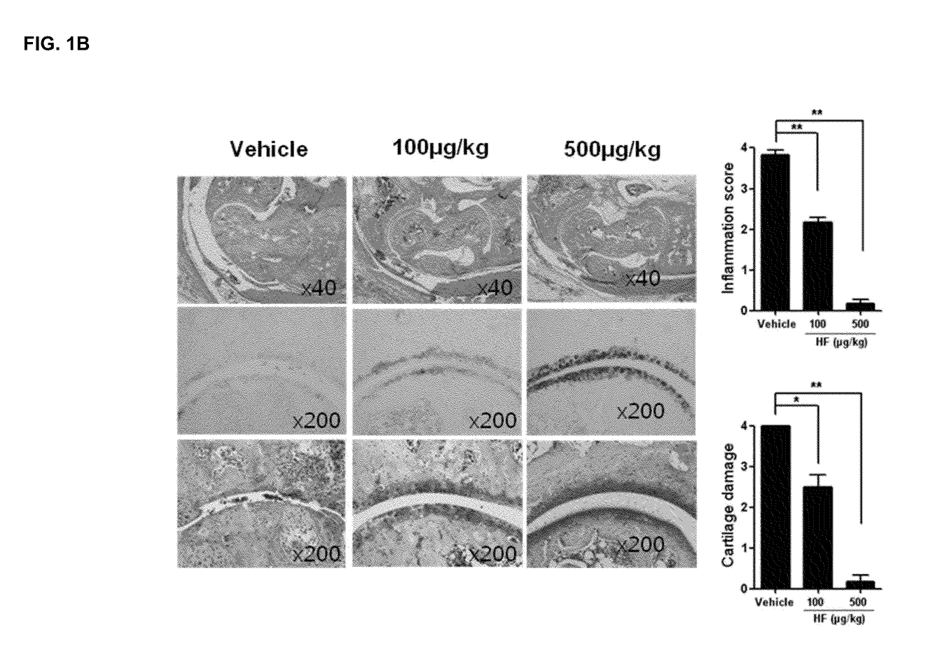

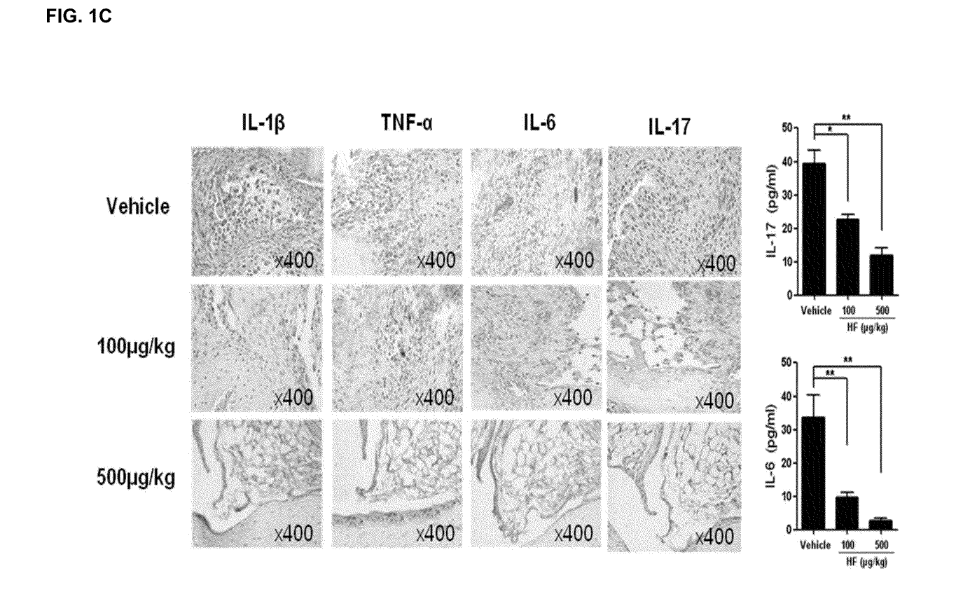

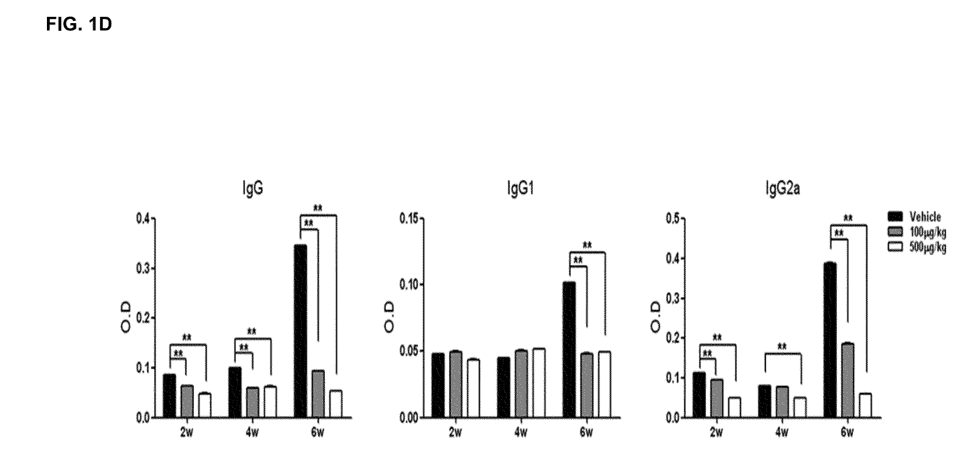

As a result, as can be seen in FIG. 1A, the experimental group treated with halofuginone showed a decrease in the arthritis index score in a dose-dependent manner. In particular, the experimental group treated with halofuginone (500 μg/kg) showed no arthritis lesion at all by the naked eye.

### <1-3> Analysis of the Degree of Joint Destruction by Immunohistochemical Staining Method

To examine the therapeutic effect of treating halofuginone of the present invention on arthritis, the mouse prepared in Example <1-1> (the mouse with arthritis injected with halofuginone) was sacrificed and the degree of joint destruction was analyzed via immunohistochemical staining method. The group treated with a solution in which DMSO was diluted in saline was used as the control group.

The immunohistochemical staining method was performed by the process shown below. The joints of the mice group used above were collected, fixed with 10% neutral buffered formalin, the bones were decalcified with EDTA and embedded in paraffin, the joint tissue was prepared into 7 μm-thick slices and attached to slides. Before performing the basic staining, the joint slices went through deparaffinization process using xylene, and dipped into ethanol from high concentration to low concentration. The staining process was performed via hematoxylin/eosin staining, and Safranin O and Toluidine Blue methods, which can sense the proteoglycan contained in cartilage, were used. The immunohistochemical staining method was used in the joints of mice and analyzed under an optical microscope.

Additionally, the inflammation index score and the degree of cartilage damage were digitized based on the tissue staining. After performing the immunohistochemical staining method, the score of inflammation index digitized the result of H & E staining, and cartilage damage digitized the results of safranin O staining and Toluidine blue staining. The standards for the digitization are as shown below.
0=no destruction
1=minimal erosion, limited to single spots
2=slight to moderate erosion in a limited area
3=more extensive erosion
4=general destruction

As a result, as can be seen in FIG. 1B, the joint destruction was shown to significantly decrease according to the concentration of halofuginone treatment. Additionally, when the expression of IL-17 and IL-6 were examined in sera of each experimental group via ELISA after staining the inflammatory cytokines within the joint tissue, the levels of the inflammatory cytokines were shown to significantly decrease by the halofuginone treatment (FIG. 1C).

### <1-4> Measurement of Type II Collagen-Specific IgG, IgG1, and IgG2a

The concentrations of murine anti-CII-specific IgG, IgG1, and IgG2a were measured by ELISA method by collecting sera from a mouse with arthritis 4 weeks after injecting the mouse with halofuginone (Bethyl Lab Co., Montgomery, Tex., USA). The group treated with a solution in which DMSO was diluted in saline was used as the control group.

The method of measuring the anti-CII-specific IgG, IgG1, and IgG2a antibodies is as follows. The CII was diluted in 0.05M sodium carbonate coating buffer (pH 9.6) at a concentration of 4 μg/mL, coated onto a 96-well microtiter plate, and placed at 4° C. for 18 hours. After removing the coated solution, for the inhibition of non-specific binding, TBS (pH 8.0) containing 1% bovine serum albumin (BSA: Amre-sco, solon, Ohio, USA) was added in an amount of 200 μL thereto and reacted at room temperature for 1 hour. For the measurement of anti-CII-specific IgG, IgG1, and IgG2a antibodies, the specimen was diluted at a 1:1,000 ratio, and in particular, TBS (pH 8.0) containing 1% BSA and 0.05% Tween 20 (Amresco) was used as the diluted solution. Then, the sera sample diluted in advance was added to each well in an amount of 50 μL and reacted at room temperature for 1 hour. Upon completion of the reaction, the TBS (pH 8.0) containing 0.05% Tween 20 (Amresco) was washed 5 times, detection antibody/HRP conjugate (anti-mouse IgG HRP) was diluted at a 1:75,000 ratio, 50 μl of the diluted conjugate was added in each of wells, and then, the conjugate was reacted at room temperature for 1 hour. After the reaction, the resultant was washed 5 times with IgG washing buffer, and allowed to develop color using TMB+H₂O₂ system (KPL, Gaithersburg, Md., USA), and an equal amount of 1 N H₂SO₄ was added thereto to stop the reaction. The absorbance of the resultant was measured at 450 nm using the ELISA reader and the result of antibody measurement was indicated by absorbance itself.

As a result, as can be seen in FIG. 1D, it was confirmed that halofuginone treatment significantly decreased the concentrations of IgG, IgG1, and IgG2a within the sera.

<1-5> Effect of Halofuginone on Th17- and Treg Cells in Splenocytes

To examine the effect of halofuginone treatment in an animal model on Th17 and Treg factors, the measurement was performed using a confocal microscope after the immunostaining. Specifically, the spleens of the mice in the experimental group and control group in Examples were collected and embedded with an OCT compound, and the tissues rapidly cooled using liquid nitrogen were sliced into a thickness of 7 μm using a cryo microtome, and attached to slides. The sliced sections attached to the slides were fixed with acetone, and non-specific binding was blocked by reacting with 10% normal goat serum for 30 minutes. The first antibodies, i.e., FITC-labeled anti-Foxp3 Ab, PElabeled anti-IL-17 Ab APC-labeled anti-CD4 Ab, Allophycocyanin-labeled anti-CD25 Ab (Biolegend), biotinylated anti-CD4 Ab (BD Biosciences, San Jose, Calif., USA), were diluted in PBS (pH 7.5) at a 1:100 ratio and reacted at 4° C. overnight. On the next day, the resultant was washed with PBS and reacted with streptavidin cy-3 at room temperature for 2 hours. The stained tissues were analyzed using a confocal microscope (LSM 510 Meta. Zeiss, Gottingen, Germany), and the number of IL-17+ cells and Foxp3+ cells were scored under a microscopic observation, and the results are shown in graphs. Meanwhile, pSTAT3 and pSTAT5 were immunostained in the same manner as described above and the measurement was performed using a confocal microscope, and in particular, PE-conjugated anti-phospho-Stat3 (Y705 and S727; 1:50), and Alexa Fluor 488-conjugated anti-phospho-Stat5 (Y694; 1:100) (all from BD Biosciences) antibodies were used.

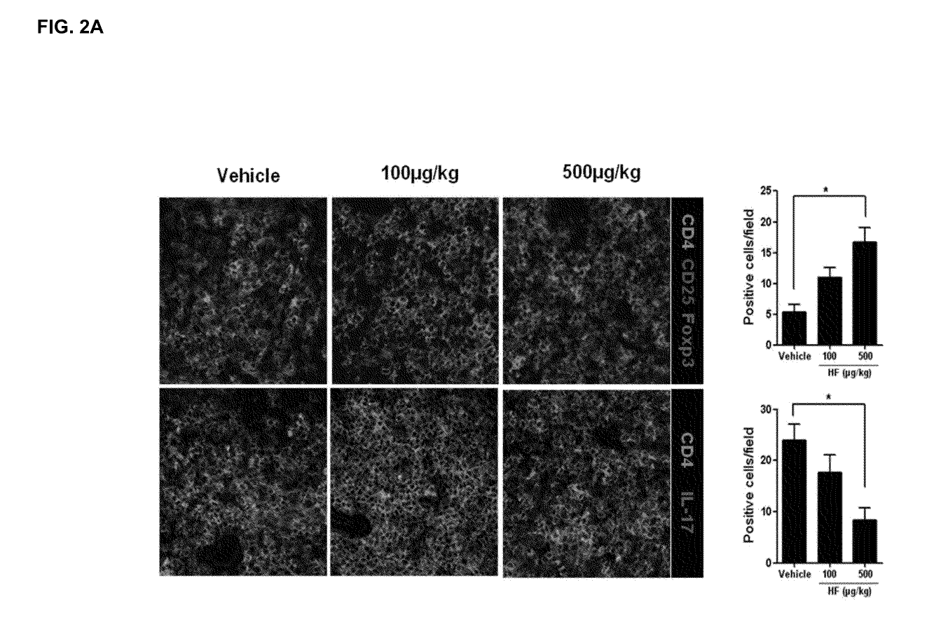

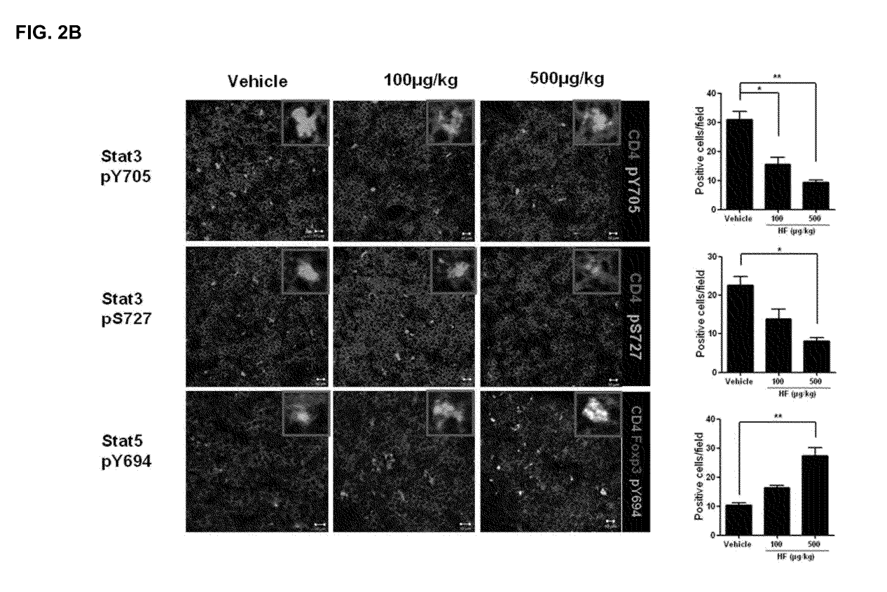

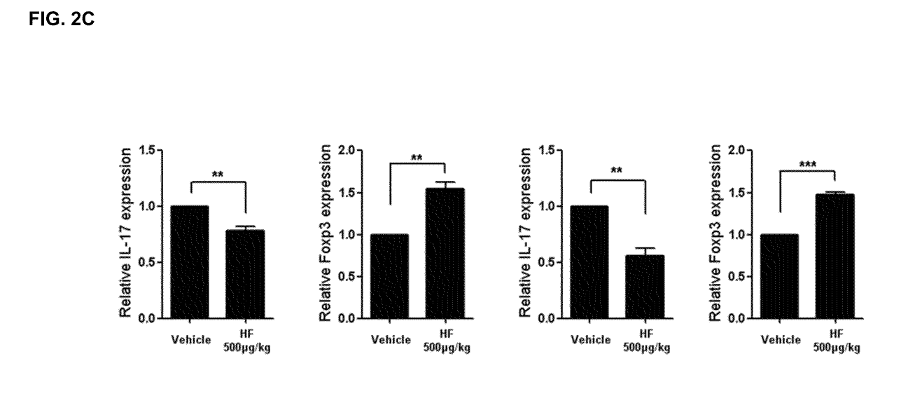

As a result, as can be seen in FIG. 2A, it was confirmed that, in the splenocytes of the experimental group injected with halofuginone, the number of Th17 cells decreased while the number of Treg cells increased (FIG. 2A). Additionally, when the expression of pSTAT3 and pSTAT5 among the CD4+ cells of the splenocytes of the experimental group injected with halofuginone were observed by immunostaining, the expression of pSTAT3 decreased while the expression of pSTAT5 increased in the splenocytes of the experimental group injected with halofuginone.

### <1-6> Expression of IL-17 and Foxp3 Genes According to Halofuginone Treatment in Splenocytes

To examine the effect of halofuginone treatment in an animal model, the RNA in the splenocytes and draining lymph nodes were extracted using the Trizol reagent, and the expression of IL-17and Foxp3 genes was confirmed by RT-PCR.

The real-time PCR was performed using the LightCycler 2.0 System Instrument and the reaction compounds were prepared by mixing 1 μL of each cDNA, 10 μL of premix Ex Taq as an enzyme for the initiation, and each primer pair to a final volume of 20 μL using distilled water. The reaction conditions were: reacted at 95° C. for 10 minutes, followed by a total of 50 cycles consisting of denaturation at 95° C. for 10 seconds, annealing at 60° C. for 5 seconds, and extension at 72° C. for 10 seconds. The values of cycle threshold (Ct) were analyzed and the amount of mRNA expression for each target was indicated in terms of the amount of beta-actin mRNA expression and the relative quantity. The primers used are shown below.

TABLE 1

| | | | SEQ |
|---|---|---|---|
| | | Base Sequence | ID |
| Primer Name | | (5′->3′) | NO |
| IL-17 | Forward | CCTCAAAGCTCAGCGTGTCC | 1 |
| | Reverse | GAGCTCACTTTTGCGCCAAG | 2 |
| Foxp3 | Forward | GGCCCTTCTCCAGGACAGA | 3 |
| | Reverse | GCTGATCATGGCTGGGTTGT | 4 |
| β-actin | Forward | GAAATCGTGCGTGACATCAAAG | 5 |
| | Reverse | TGTAGTTTCATGGATGCCACAG | 6 |

As a result, as can be seen in FIG. 2C, it was confirmed that the expression of IL-17 was decreased while the expression of Foxp3 increased in the experimental group injected with halofuginone.

<1-7> Evaluation of Osteoclast Differentiation Potential in the joint of an Animal Model with Arthritis by TRAP Staining

To examine the osteoclast differentiation potential in the joint of the animal model with arthritis by TRAP staining, the marker enzyme of osteoclasts were subjected to TRAP staining using the leukocyte acid phosphatase Kit (387-A, Sigma, St. Louis, Mo., USA) according to the manufacturer's manual. The cells having at least 5 nuclei among the TRAP positive cells (red color) were regarded as osteoclasts.

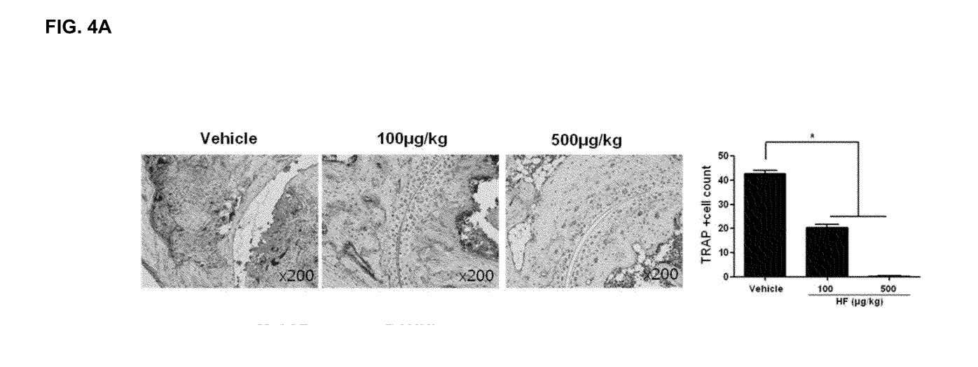

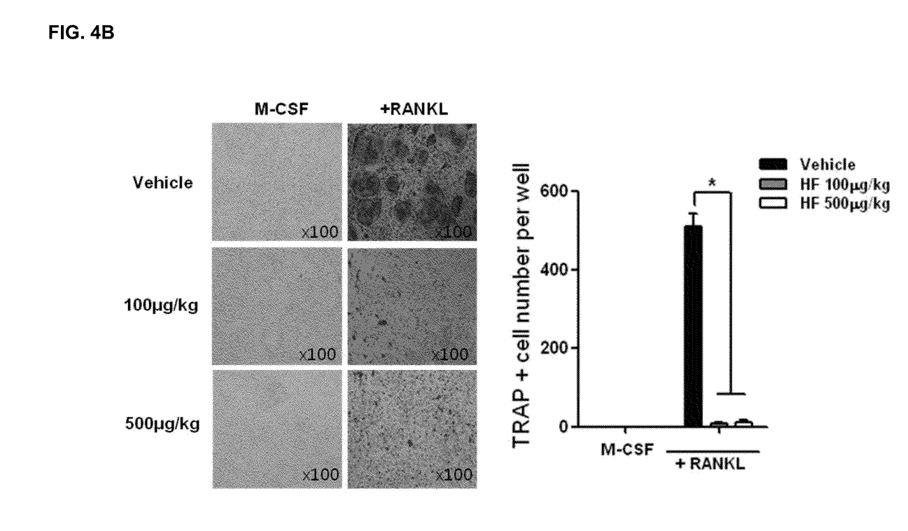

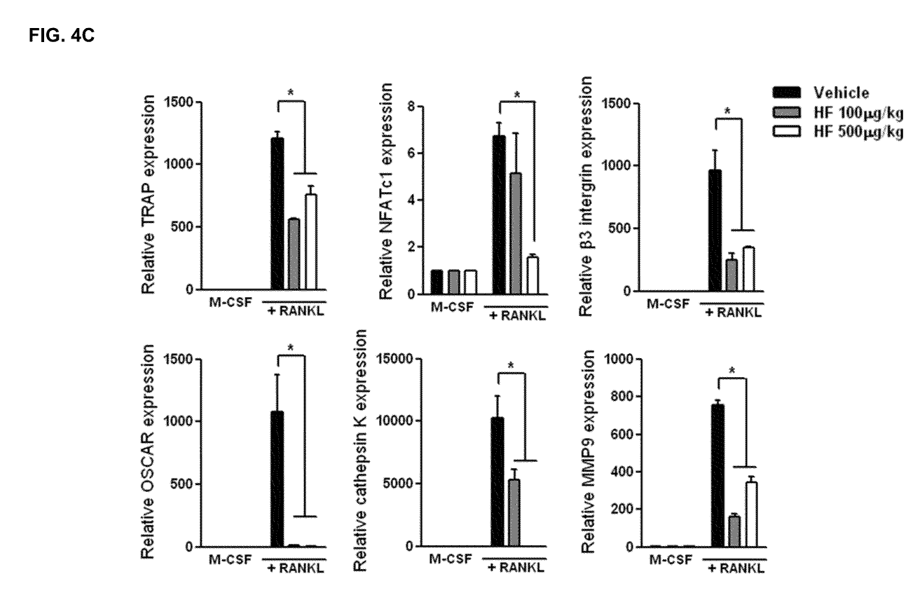

As a result, as can be seen in FIG. 4A, it was confirmed that the number of TRAP positive cells was decreased in the experimental group treated with halofuginone.

<1-8>0 Evaluation of Osteoclast Differentiation Potential in the Joint of an Animal Model with Arthritis by TRAP Staining

Accordingly, the present inventors isolated bone marrow cells from the joint of the animal model with arthritis, cultured the cells under the stimulation of M-CSF and RANKL for 7 days, and subjected them to TRAP staining using the leukocyte acid phosphatase Kit (387-A, Sigma, St. Louis, Mo., USA) according to the manufacturer's manual.

As a result, as can be seen in FIG. 4B, the number of TRAP positive cells was decreased in the experimental group treated with halofuginone, and thus it was confirmed that halofuginone has the effect of inhibiting the differentiation of bone marrow cells into osteoclasts.

<1-9> Analysis of Expression of Factors Associated with the Differentiation of Osteoclasts in the Bone Marrow Cells of the Joint of an Animal Model with Arthritis

Finally, to examine how the expression of the factors associated with the differentiation of osteoclasts in the bone marrow cells in Example <1-8>, the present inventors performed the real-time PCR in the same manner as suggested in Example <1-6>. The primers used are shown below.

TABLE 2

| | | | SEQ |
|---|---|---|---|
| | | Primer Sequence | ID |
| Primer Name | | (5′->3′) | NO |
| TRAP | Forward | TCCTGGCTCAAAAAGCAGTT | 7 |
| | Reverse | ACATAGCCCACACCGTTCTC | 8 |
| NFATc1 | Forward | CGGGAAGAAGATGGTGCTGT | 9 |
| | Reverse | TTGGACGGGGCTGGTTAT | 10 |
| integrin β3 | Forward | CCACACGAGGCGTGAACTC | 11 |
| | Reverse | CTTCAGGTTACATCGGGGTGA | 12 |
| OSCAR | Forward | CCTAGCCTCATACCCCCAG | 13 |
| | Reverse | CAAACCGCCAGGCAGATTG | 14 |
| Cathepsin K | Forward | CAGCAGAGGTGTGTACTATG | 15 |
| | Reverse | GCGTTGTTCTTACTTCGAGC | 16 |
| MMP9 | Forward | CTGTCCAGACCAAGGGTACAGCCT | 17 |
| | Reverse | GAGGTATAGTGGGACACATAGTGG | 18 |

As a result, as can be seen in FIG. 4C, the mRNA expression levels of TRAP, NFATc1, b3 integrin, OSCAR, cathepsin K, and MMP9, which are associated with the differentiation of osteoclasts were increased by the RANKL treatment but the expression levels were all decreased by the halofuginone treatment.

### EXAMPLE 2

### Analysis of Therapeutic Effect of a Halofuginone Compound on the Treatment of Bone Diseases Via Inhibition of Differentiation of Osteoclasts

### <2-1> Observation of Osteoclast Differentiation in a Mouse Animal Model

For test animals, 6 to 7-week old male DBA-1 mice were used. The differentiation of osteoclasts in bone marrow cells was observed in a monoculture system. Bone marrow cells were isolated from the tibia and the femur of mice, and the RBC of the isolated bone marrow cells were lysed, inoculated into a 24-well plate after diluting in 10% Minimum α-MEM to a concentration of 2 [symbol] 10⁵ cells/well, and cultured for 12 hours. The adherent cells were removed, stimulated with a macrophage-colony stimulating factor (M-CSF) and cultured for 3 days, and treated with M-CSF (10 ng/mL), receptor activator of NF-κB ligand (RANKL; 50 ng/mL), and halofuginone and cultured for 2 days. The medium was replaced in 2 days, retreated in the same manner, and cultured for 2 days.

### <2-2> Differentiation of Th17-, Th1-, and Th2 Cells by Halofuginone Treatment

Splenocytes were obtained from mice, that is, the spleens removed from the mice and the spleen tissues were thinly ground using a tearing slide and the red blood cells (RBC) in the splenocytes were removed with the RBC lysis solution. Then, PBS buffer solution was added thereto, and the mixture was centrifuged, and washed to obtain splenocytes. Then, the singe cells (1 [symbol] 10⁶) isolated from the spleens were aliquoted into a 24-well plate coated with anti-CD3 antibody (1 μg/mL) were treated under the condition of capable of stimulating Th17 cells (anti-CD28 antibody (1 μg/mL), TGF-β (2 ng/ mL), IL-6 (20 ng/mL), anti-IL-4 (10 μg/mL), and anti-IFN-γ (10 μg/mL); the condition of capable of stimulating Th1 cells (anti-CD28 antibody (1 μg/mL), IL-12 (10 ng/mL), and anti-IL-4 (10 μg/mL)); or the condition of capable of stimulating Th2 cells (anti-CD28 antibody (1 μg/mL), IL-4 (10 ng/mL), and anti-IFN-γ (10 μg/mL)); and the cells were cultured for 3 days, and in particular, cultured after treating the cells with halofuginone at respective concentration and cultured. Specifically, DMSO treatment was used for the control group.

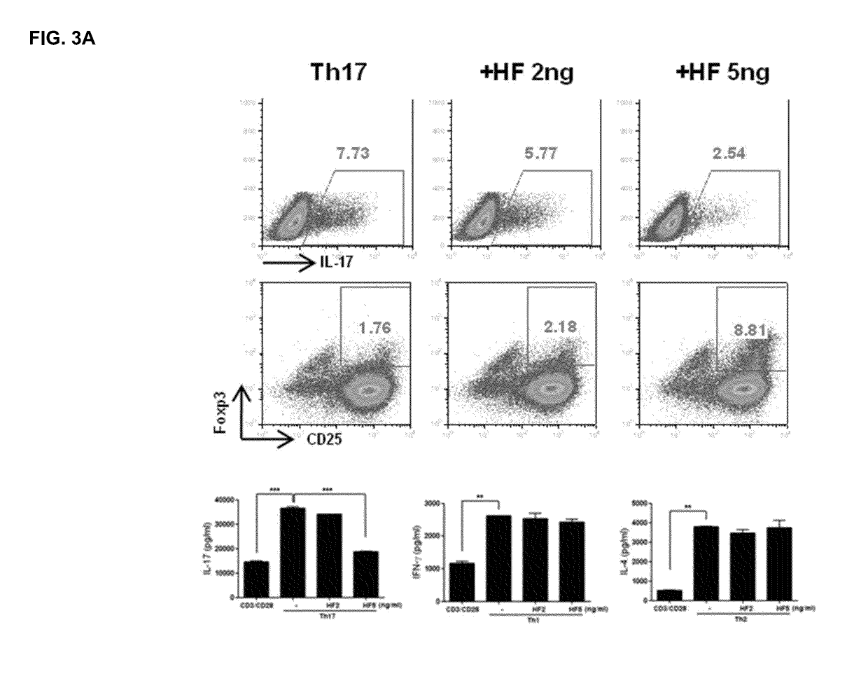

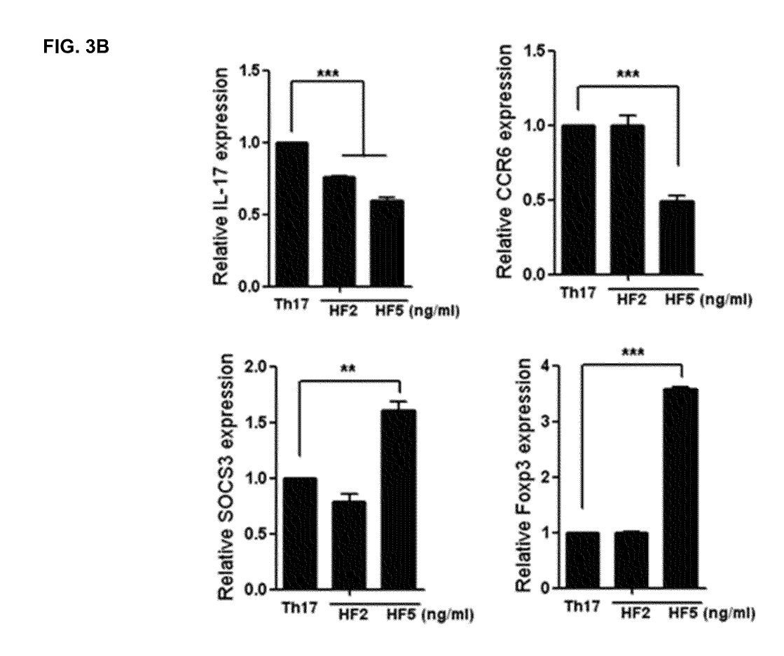

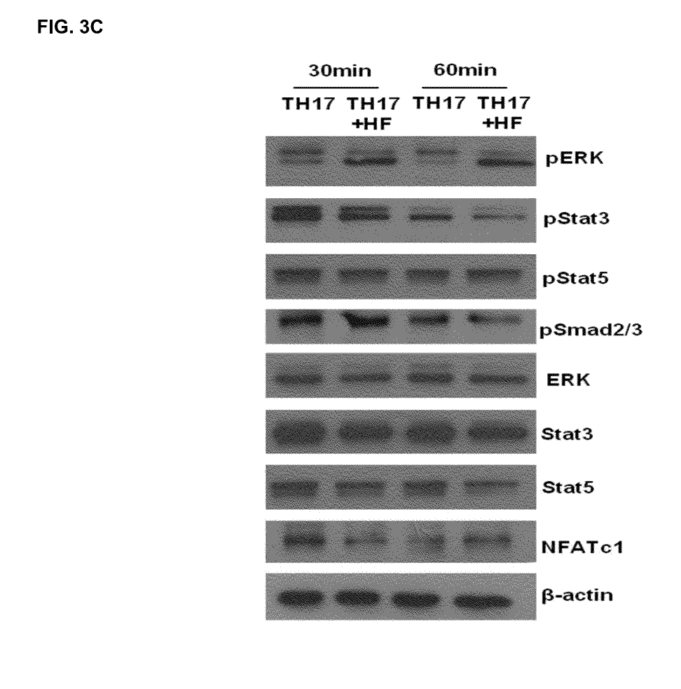

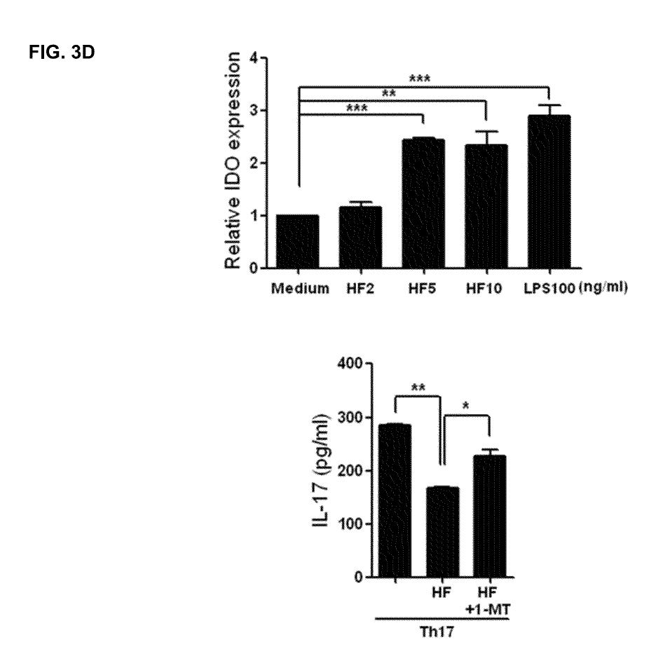

As a result, as can be seen in FIG. 3A, when the cells were subjected to halofuginone treatment under the condition of capable of differentiating Th17 cells, Th17 cells were inhibited while the Treg cells increased. However, when the cells were subjected to halofuginone treatment under the conditions of capable of differentiating Th1 cells, IFN-gamma, and Th2 cells, the halofuginone treatment did not affect the production of IL-4.

<2-3> Observation of the Amount of mRNA Expression of IL-17, CCR6, SOCS3, and Foxp3 in Th17 Cells According to Halofuginone Treatment

The expression of IL-17, CCR6, SOCS3, and Foxp3 in the cells under the condition of Example <2-2> was analyzed by real-time PCR as described in Example <1-6>. The primers used are shown below.

TABLE 3

| | | | SEQ |
|---|---|---|---|
| | | Primer Sequence | ID |
| Primer Name | | (5′->3′) | NO |
| CCR6 | Forward | CCTCACATTCTTAGGACTGGAGC | 19 |
| | Reverse | GGCAATCAGAGCTCTCGGA | 20 |
| SOCS3 | Forward | CCTTTGACAAGCGGACTCTC | 21 |
| | Reverse | GCCAGCATAAAAACCCTTCA | 22 |

As a result, as can be seen in FIG. 3B, it was confirmed that halofuginone inhibits the expression of IL-17 and CCR6 while increasing the expression of SOCS and Foxp3.

<2-4> Analysis of pERK Expression in Th17 Cells According to Halofuginone Treatment

To examine whether halofuginone activates the ERK of Th17 cells, western blotting was performed for pERK in the cells under the condition of Example <2-1>.

To this end, the Th17 cells were lysed in a lysis buffer [20 mM Tris-HCl (pH 7.5), 150 mM NaCl, 1% Triton X-100, 1 mM EDTA, 1 mM EGTA, 1 mM phenylmethylsulfonyl fluoride, 10 mM β-glycerophosphate, 1 mM NaF, 1 mM Na₃VO₄, and 19 Protease Inhibitow Cocktail™ (Roche Molecular Biochemicals, Indianapolis, Ind., USA)]. From the lysate, an equal amount of the protein (about 20 μg) was separated by sodium dodecyl sulfate-polyacrylamide gel electrophoresis and then transferred to a nitrocellulose membrane. Then, the blots were probed with NFATc1 and phospho-Smad2/3 (all from Santa Cruz Biotechnology), Stat3, Stat5, ERK, phospho-Stat3, phospho-stat5, and phospho-ERK (all from Cell Signaling Technology), and β-actin (Sigma) antibodies, and confirmed by ECL (Amersham, Del., USA).

As a result, as can be seen in FIG. 3C, it was confirmed that the expression of pERK increased in the Th17 cells stimulated with halofuginone.

### <2-5> Measurement of the Amount of IL-17 Cytokines via ELISA

The dendritic cells cultured for 2 days after stimulating with halofuginone and the T cells cultured for 2 days under the condition of differentiating Th17 cells of <2-2> were co-cultured for 2 additional days, and IL-17 was measured using the supernatant by ELISA.

That is, the supernatants of the cell cultures were collected and the amount of IL-17 was measured via sandwich ELISA method. To this end, first, the 96-well plate was treated with monoclonal anti-IL-17 (2 g/mL) and reacted at 4° C. overnight, and upon reaction, non-specific binding was blocked using a blocking solution (1% BSA/PBST). Then, IL-17 in a half amount was diluted continuously and used as standards, and the supernatants of cell cultures were reacted at room temperature for 2 hours. Then, the biotinylated anti-IL-17 were reacted at room temperature for 2 hours, washed 4 times, and added with ExtraAvidin-alkaline phosphatase conjugate after dilution, and reacted at room temperature for 2 hours. Subsequently, PNPP/DEA solution was added thereto to develop color and the absorbance was measured at 405 nm.

As a result, as can be seen in FIG. 3D, the T cells, which were co-cultured with the dendritic cells stimulated with halofuginone, showed a decrease in the production of IL-17, however, the T cells which were co-cultured with the dendritic cells co-stimulated with 1-MT (an IDO inhibitor) and halofuginone showed no decrease in the production of IL-17 of T cells.

### <2-6> Analysis of the Effect of Halofuginone on the Cell Cycle of Osteoclast Differentiation

Murine bone marrow cells were differentiated for 5 days by stimulating with M-CSF, RANKL, and halofuginone and subjected to TRAP staining according to the method described in Example <1-8> to confirm the number of TRAP positive cells. Additionally, the expression of c-Fos, JunB, and Jpd2 in the bone marrow cells were confirmed by real-time PCR according to the method described in Example <1-6>.

TABLE 4

| | | | SEQ |
|---|---|---|---|
| | | Primer Sequence | ID |
| Primer Name | | (5′->3′) | NO |
| c-Fos | Forward | CCATGATGTTCTCGGGTTTC | 23 |
| | Reverse | TGTCACCGTGGGGATAAAGT | 24 |
| JunB | Forward | ATGTGCACGAAAATGGAACA | 25 |
| | Reverse | CCTGACCCGAAAAGTAGCTG | 26 |
| Jpd2 | Forward | CGCTGACATCCGCAACATT | 27 |
| | Reverse | GGCCTCTTGCCCAGTTTCA | 28 |

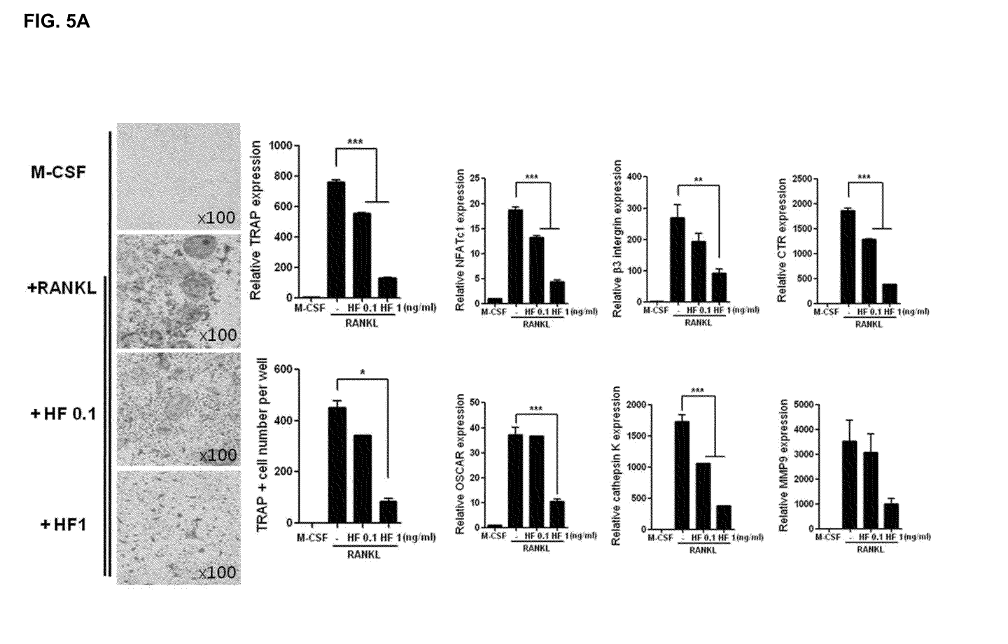

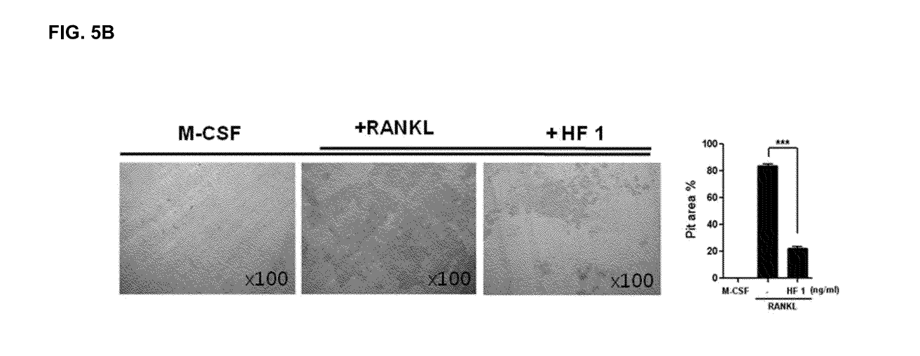

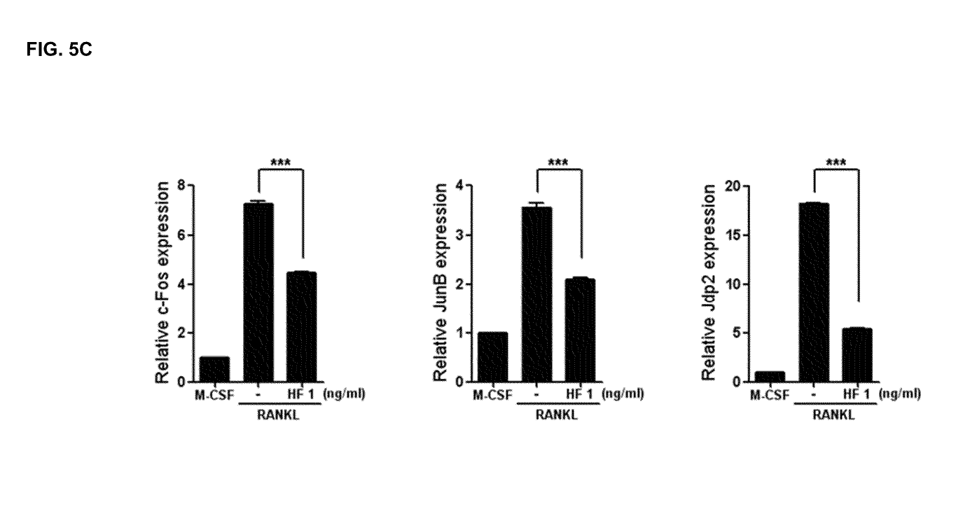

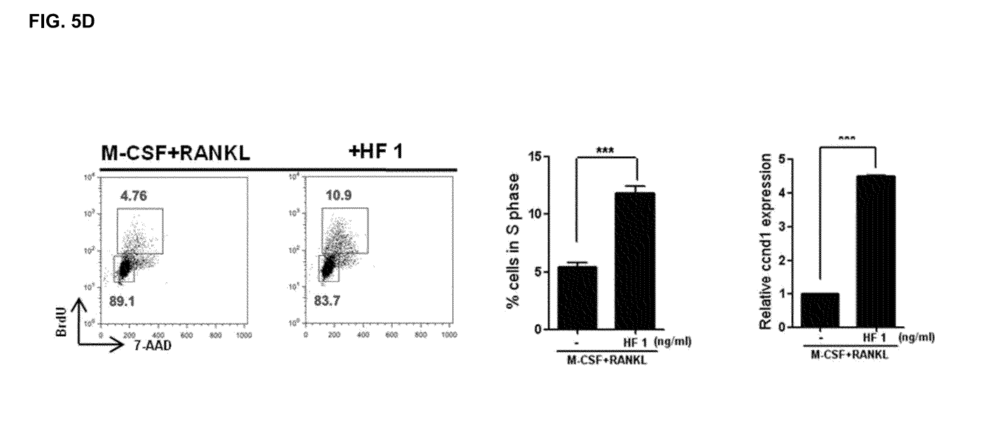

As a result, as can be seen in FIG. 5A and FIG. 5C, it was confirmed that halofuginone inhibits the expression of factors associated with the differentiation of osteoclasts and also inhibits the mRNA expression of c-Fos, JunB, and Jpd2.

Meanwhile, to examine the effect of halofuginone on cell cycle during the process of osteoclast differentiation, osteoclasts were subjected to 7-AAD and BrdU staining.

As a result, as can be seen in FIG. 5D, it was confirmed that when being stimulated with halofuginone, the progress from G0/G1 into S phase was increased during the process of osteoclast differentiation, and also that the expression of ccnd1 encoding Cyclin D1, which regulates cell cycle, was increased.

### <2-7> Analysis of the Effect of Halofuginone on the Bone Resorption Potential by Osteoclasts

To examine the effect of halofuginone on the bone resorption potential by osteoclasts, murine bone marrow cells were cultured in Dentine slice, and the cultured cells were stimulated with M-CSF, RANKL, and halofuginone, and cultured for 21 additional days. Then, the cells were removed using 5% sodium hypochlorite solution, subjected to Hematoxylin staining, and the bone resorption lacunae formed by the bone resorption of osteoclasts were photographed.

As a result, as can be seen in FIG. 5B, it was confirmed that the bone resorption potential was significantly reduced by halofuginone.

### <2-8> Analysis of Expression of IL-17 and Foxp3 by Flow Cytometer in Human T Cells.

The CD4 T cells isolated from human peripheral blood were treated with halofuginone under the condition of differentiating Th17 cells (anti-CD3 (0.5 μg/mL), anti-CD28 (0.5 μg/mL), anti-IFN-γ (10 μg/mL), anti-IL-4 (10 μg/mL), IL-6 (20 ng/mL), and IL-1β (10 ng/mL)) and cultured. Then, the cells were collected, washed with the FACs buffer for flow cytometry analysis, subjected to blocking at 4° C. for the inhibition of non-specific binding for 15 minutes, added with CD4 and CD25 (i.e., cell surface markers) along with anti-CD4-PerCP and anti-CD25-APC and reacted at 4° C. for 30 minutes, and washed with a perm wash buffer. After performing the Cytofix/cytoperm process at 4° C. for 20 minutes, the resultant was washed with the perm wash buffer. Anti Foxp3-FITC and anti IL-17 PE were added thereto, reacted at 4° C. for 30 minutes, and washed with the perm wash buffer. Upon completion of staining, the cells were analyzed using the fluorescentactivated cell sorter (FACs) regarding the expression levels of IL-17 cytokines and Foxp3 according to the halofuginone treatment.

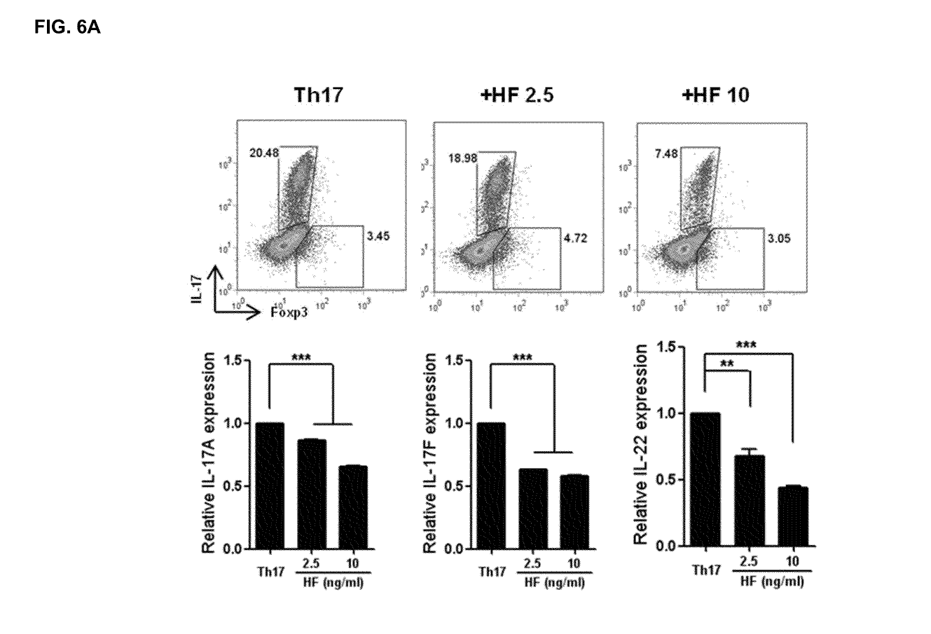

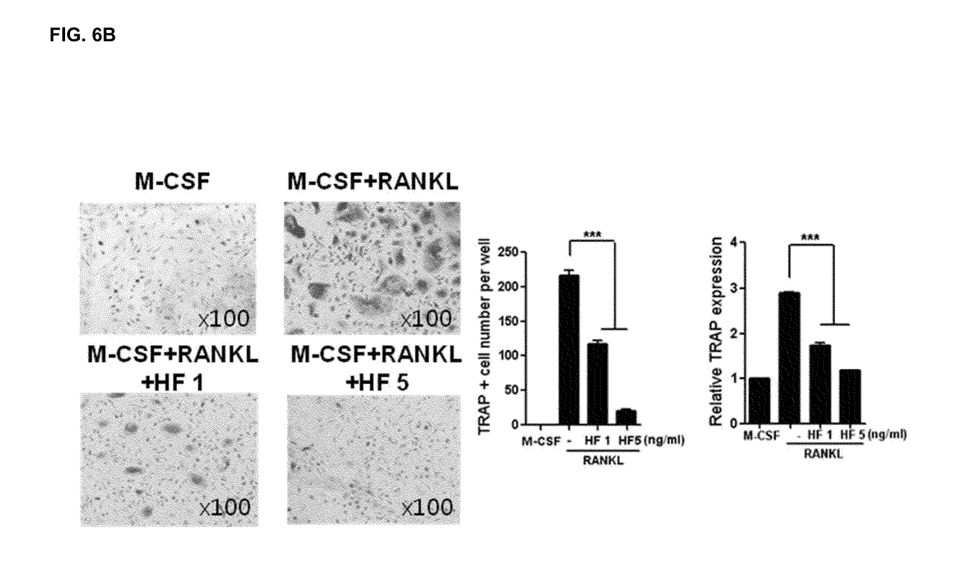

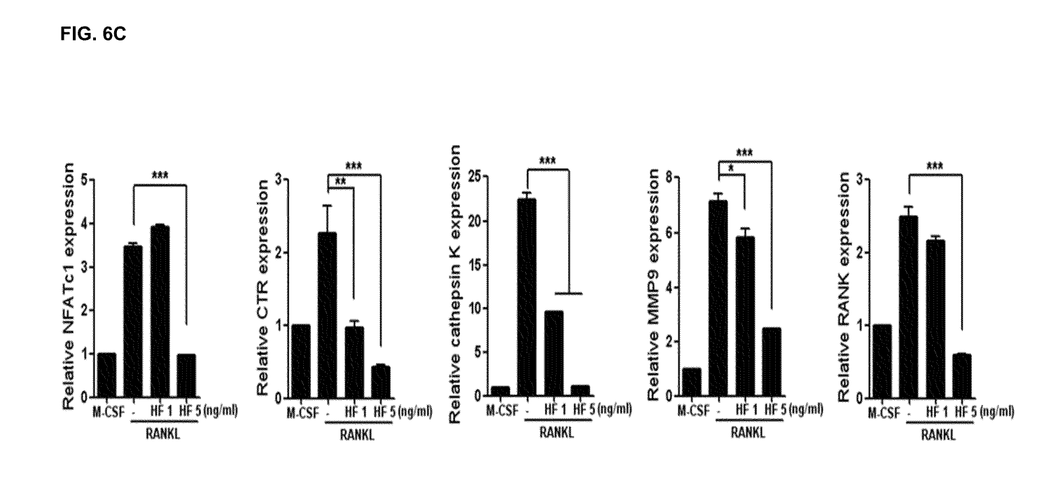

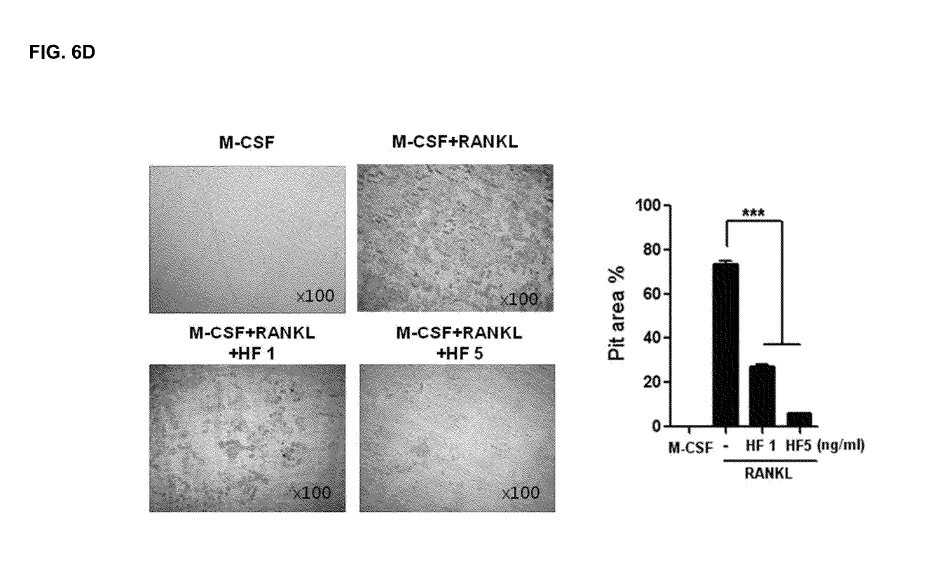

As a result, as can be seen in FIG. 6A, it was confirmed that halofuginone reduces the number of Th17 cells while increasing the number of Treg cells.

### <2-9> Observation of the Differentiation of Osteoclasts in Mononuclear Cells of Human Peripheral Blood

The mononuclear cells of human peripheral blood were cultured for 9 days after stimulating with M-CSF, RANKL, and halofuginone. Then, the cells were stained by TRAP according to the manufacturer's manual, and the amount of mRNA of TRAP, NFATc1, calcitonin receptor (CTR), cathepsin K, MMP9, and RANK were confirmed by real-time PCR.

TABLE 5

| | | | SEQ |
|---|---|---|---|
| | | Primer Sequence | ID |
| Primer Name | | (5′->3′) | NO |
| TRAP | Forward | GACCACCTTGGCAATGTCTCTG | 29 |
| | Reverse | TGGCTGAGGAAGTCATCTGAGTTG | 30 |
| NFATc1 | Forward | GCATCACAGGGAAGACCGTGTC | 31 |
| | Reverse | GAAGTTCAATGTCGGAGTTTCTGAG | 32 |
| CTR | Forward | TGGTGCCAACCACTATCCATGC | 33 |
| | Reverse | CACAAGTGCCGCCATGACAG | 34 |
| Cathepsin K | Forward | TGAGGCTTCTCTTGGTGTCCATAC | 35 |
| | Reverse | AAAGGGTGTCATTACTGCGGG | 36 |
| MMP9 | Forward | TGGGGGGCAACTCGGC | 37 |
| | Reverse | GGAATGATCTAAGCCCAG | 38 |
| RANK | Forward | GCTGTAACAAATGTGAACCAGGA | 39 |
| | Reverse | GCCTTGCCTGTATCACAAACT | 40 |
| β-actin | Forward | GGACTTCGAGCAAGAGATGG | 41 |
| | Reverse | TGTGTTGGGGTACAGGTCTTTG | 42 |

As a result, as can be seen in FIGS. 6B and 6C, the halofuginone treatment decreased the mRNA expression of TRAP, NFATc1, calcitonin receptor(CTR), cathepsin K, MMP9, and RANK in a dose-dependent manner, and thus it was confirmed that halofuginone can effectively inhibit the differentiation of osteoclasts.

### <2-10> Analysis of the Effect of Halofuginone on the Bone Resorption Potential by Osteoclasts in Human Cells

The effect of halofuginone on the bone resorption potential by osteoclasts was observed using the mononuclear cells of human peripheral blood, in the same manner as described in Example <2-7>.

As a result, as can be seen in FIG. 6D, the bone resorption potential was significantly reduced by the halofuginone treatment.

### EXAMPLE 3

### Analysis of the Effect of a Halofuginone Compound on the Promotion of the Differentiation of Osteoblasts

Furthermore, to examine whether the halofuginone compound of the present invention can promote the differentiation and the activity of osteoblasts, the present inventors performed the following experiment for the purpose of confirming the effect of the halofuginone compound on the expression and the activity of Rcan3 and Sox6, which are factors associated with the differentiation of osteoblasts. Specifically, the DBA/1J mice were subjected to the primary immunization by intradermally injecting bovine type II collagen (hereinafter, “CII”) in an amount of 100 μL, and one week thereafter, injected intraperitoneally with halofuginone (500 μg/kg). Fifty days thereafter, the mice were sacrificed, and the expression levels of Rcan3 and Sox6 in the joint tissue of the mice were confirmed via immunohistochemical staining method. The group of mice not treated with halofuginone was used as the control group.

The immunohistochemical staining method was performed as follows. The joints of the mice group used above were collected, fixed with 10% neutral buffered formalin, the bones were decalcified with Calci-Clear Rapid bone decalcifier and embedded in paraffin, the joint tissue was prepared into 7 μm-thick slices and attached to slides. Before performing the basic staining, the joint slices went through deparaffinization process using xylene, and dipped into ethanol from high concentration to low concentration. Additionally, for sensing the expression levels of Rcan3 and Sox6, an immunostaining was performed using antibodies to Sox-6 (Santa Cruz Biotechnology, sc-20092) and Calcipressin 3 (abcam, ab38312) and the expression levels of the proteins were observed via analysis under an optical microscope.

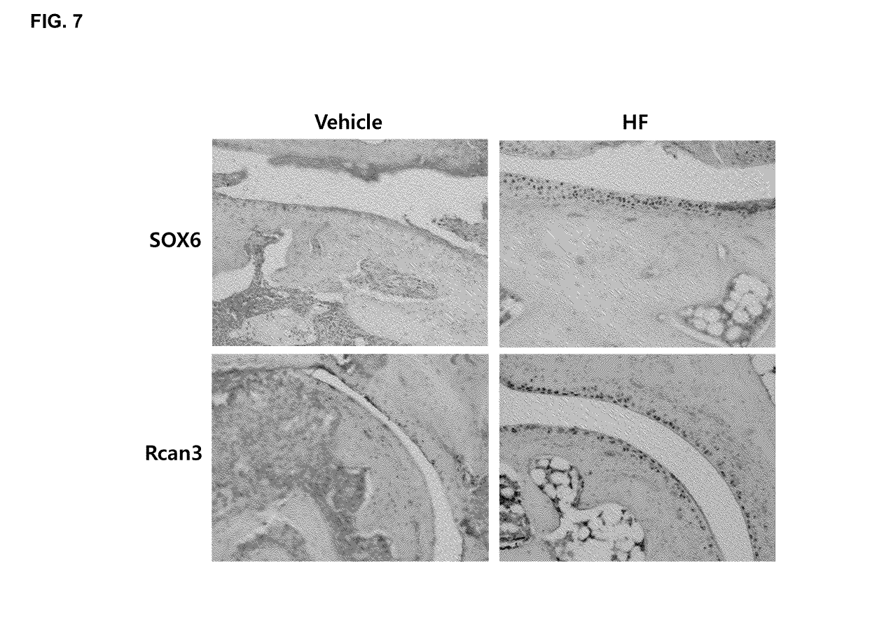

As a result of the analysis, as shown in FIG. 7, it was confirmed that the group treated with halofuginone showed a significant increase in the expression of Rcan3 and Sox6, which are related to the differentiation and development of osteoblasts, compared to the group not treated with halofuginone.

Accordingly, based on the results described above, the present inventors have confirmed that halofuginone has the effects of promoting the differentiation and the development of osteoblasts while inhibiting the differentiation of osteoclasts, and conclusively, have confirmed that halofuginone can be used as a novel therapeutic agent for the treatment of bone diseases.

The present invention has been explained referring to exemplary embodiments. Those of ordinary skill in the art will recognize that the present invention may be embodied in other specific forms without departing from its spirit or essential characteristics. The described embodiments are to be considered in all respects only as illustrative and not restrictive. The scope of the present invention is, therefore, indicated by the appended claims rather than by the foregoing description. All changes which come within the meaning and range of equivalency of the claims are to be embraced within the scope of the present invention.

The present invention was performed being sponsored by the National Research Development Business Program described below.

1. Project No: HI09C1555

2. Name of Research Business: Health and Medical Treatment R&D Business

3. Name of Research Project: Antibody Production by Control of Th17 Cell Group in Solid Organ Transplantation, Control of Immune Response and Development of Immunological Tolerance

4. Agency in Charge: Industry-Academic Cooperation Foundation, the Catholic University of Korea

5. Government Department: Ministry of Health and Welfare

6. National R&D Management Agency: Korea Health Industry Development

Institute

7. Research Period: Dec. 1, 2013 to Nov. 30, 2014

1. Project No: HI13C0016

2. Name of Research Business: Health and Medical Treatment R&D Business, the 2^{nd} Half of 2014 (Development of Technologies for Disease Treatment_Rare Disease Translational Research Center)

3. Name of Research Project: Establishment of Shogren animal system and Disease-Specific Diagnosis and Treatment Targets, Discovery of Novel Materials

4. Agency in Charge: Industry-Academic Cooperation Foundation, the Catholic University of Korea

5. Government Department: Ministry of Health and Welfare

6. National R&D Management Agency: Korea Health Industry Development

Institute

7. Research Period: Jun. 1, 2014 to May 31, 2015

### INDUSTRIAL APPLICABILITY

The composition of the present invention for treating or preventing bone diseases containing halofuginone as an active ingredient has the effects of inhibiting osteoclast differentiation by reducing the expression of Th17 cells while increasing the expression of Treg cells and also the effect of effectively inhibiting the bone resorption by osteoclasts, thus being expected to be effectively used for the treatment or prevention of bone diseases.

1.-14. (canceled)

15. A method of treating or preventing a bone disease, the method comprising administering an effective amount of a halofuginone compound or a salt thereof to a subject.

16. The method of claim 15, wherein the bone disease is any one selected from the group consisting of osteoporosis, osteoarthritis, osteopetrosis, Paget's disease, osteomalacia, rickets, ossifying fibroma, adynamic bone diseases, metabolic bone diseases, and rheumatoid arthritis, which is a bone-destroying disease through bone damage caused by bone metastasis of cancer cells and immune inflammatory response.

17. The method of claim 15, wherein the halofuginone exhibits a therapeutic effect by reducing or inhibiting osteoclast differentiation.

18. The method of claim 17, wherein the halofuginone exhibits a therapeutic effect by reducing or inhibiting osteoclast differentiation induced by RANKL.

19. The method of claim 17, wherein the halofuginone reduces or inhibits osteoclast differentiation by the inhibition of Th17 expression.

20. The method of claim 19, wherein the halofuginone inhibits the expression of a Th17 cell by activating ERK in the Th17 cell.

21. The method of claim 15, wherein the halofuginone exhibits a therapeutic effect by inhibiting the bone resorption by osteoclasts.

22. The method of claim 15, wherein the halofuginone promotes the differentiation or development of osteoblasts.

23. The method of claim 15, wherein the halofuginone compound is administered to a subject in an amount of from 0.5 g/kg to 2000 g/kg.

24. A method for promoting the differentiation or activity of osteoblasts, the method comprising administering an effective amount of a halofuginone compound or a salt thereof to a subject.

25. The method of claim 24, wherein the halofuginone promotes the expression or activity of SOX6 or Rcan3, which is a factor associated with osteoblast differentiation.

26. A method for inhibiting osteoclast differentiation, the method comprising administering an effective amount of a halofuginone compound or a salt thereof to a subject.
